(19) United States
(12) Patent Application Publication
(10) Pub. No.: US 2008/0268054 A1
(43) Pub. Date: Oct. 30, 2008

(54) DERMAL DERIVED HUMAN STEM CELLS AND COMPOSITIONS AND METHODS THEREOF

(76) Inventors: Eugene Bell, Boston, MA (US); Vladimir Russakovsky, West Roxbury, MA (US); Naheed Banu, Brookline, MA (US); Nuiliant Bell, Boston, MA (US)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 12/053,435

(22) Filed: Mar. 21, 2008

Related U.S. Application Data
(63) Continuation-in-part of application No. 11/610,021, filed on Dec. 13, 2006.
Application No. 11/610,021 is a continuation of application No. 10/430,041, filed on May 5, 2003, now abandoned.
Application No. 10/430,041 is a continuation of application No. 09/901,786, filed on Jul. 9, 2001, now abandoned.
Application No. 09/901,786 is a continuation-in-part of application No. 11/678,143, filed on Feb. 23, 2007, now abandoned.
Application No. 11/678,143 is a continuation of application No. 10/400,753, filed on Mar. 27, 2003, now abandoned.
Application No. 10/400,753 is a continuation of application No. 10/005,053, filed on Dec. 4, 2001, now abandoned.
(60) Provisional application No. 60/256,614, filed on Dec. 18, 2000.
(60) Provisional application No. 60/251,125, filed on Dec. 4, 2000.
(60) Provisional application No. 60/256,593, filed on May 29, 2001.
(60) Provisional application No. 60/251,125, filed on Dec. 4, 2000.
(60) Provisional application No. 60/907,131, filed on Mar. 22, 2007.
(60) Provisional application No. 60/924,729, filed on May 29, 2007.

(51) Int. Cl. A61K 9/14; C12N 5/06; A61P 43/00; A61K 35/12

(52) U.S. Cl. 424/484; 435/377; 424/93.7; 435/366

(57) ABSTRACT

This application discloses Dermal Derived Human Stem Cells (DDhSCs) and methods of making and using thereof. More specifically, the invention relates to DDhSCs derived from subsets of dedifferentiated dermal fibroblasts that can give rise to a series of cell lineages. The DDhSCs may be used, for example, in cell therapy and in the search for and development of novel medicaments.

### CROSS-REFERENCES TO RELATED APPLICATIONS

This application is a continuation-in-part of U.S. Application No. 11/610,021, which is a continuation of U.S. application Ser. No. 10/430,041, filed May 5, 2003, which is a continuation of U.S. application Ser. No. 09/901,786, filed Jul. 9, 2001, which claims to U.S. provisional application Ser. No. 60/256,614, filed Dec. 18, 2000; U.S. provisional application Ser. No. 60/256,593, filed Dec. 18, 2000; and U.S. provisional application Ser. No. 60/251,125, filed Dec. 4, 2000, all of which are herein incorporated by reference in their entireties. This application is a continuation-in-part of U.S. application No. 11/678,143, filed Feb. 23, 2007, which is a continuation of 10/400,753, filed Mar. 27, 2003, which is a continuation of U.S. application No. 10/005,053, filed Dec. 4, 2001, which claims priority to provisional application 60/251,125, filed Dec. 4, 2000, all of which are herein incorporated by reference in their entireties. This application claims the benefit of priority to U.S. Provisional Application No. 60/907,131, filed Mar. 22, 2007 and U.S. Provisional Application No. 60/924,729, filed May 29, 2007, the disclosures thereof are incorporated by reference herein in their entireties.

### FIELD OF THE INVENTION

The present invention relates to Dermal Derived Human Stem Cells (DDhSCs) and compositions of DDhSCs and their use in the augmentation of body tissues and other cell-based therapies.

### BACKGROUND

Embryonic stem cells (ESCs) are stem cells derived from the inner cell mass of a early stage embryo known as a blastocyst. Embryonic stem cells are pluripotent, meaning they are able to differentiate into all derivatives of the three primary germ layers:

ectoderm, endoderm and mesoderm. That is, ESCs may potentially develop into each of the more than 200 cell types of the adult body when given the sufficient and necessary stimulation for a specific cell type. Pluripotency distinguishes ESCs from multipotent progenitor cells found in adult, which may form a more limited number of different cell types.

The blastocyst is the structure formed in early mammalian embryogenesis, and possesses an inner cell mass and an outer cell mass. The former is the source of embryonic stem cells. The inner cell mass results from 7-8 divisions of the starting inner cell mass cell, creating a population of about 128 to 250 cells, which are the pluripotent stem cells from which all parts of the organism develop, except the extra-embryonic membranes. Under certain conditions in vitro, these cells can be kept in cycle indefinitely. At any time however, it is possible with the appropriate manipulations of the molecular environment available to the cells, to activate their developmental capacity specifically so that they differentiate along a particular pathway to become a specific phenotype. At the present time, stem cell colonies induced to become embryoid bodies develop as a random mix of phenotypes. The stem cell-like potential for fetal and adult cells endowed with pluripotential capabilities still needs to be probed.

The inner cell mass gives rise to the three germ layers of the embryo from which the complete organism develops. The three germ layers, ectoderm, mesoderm and endoderm are formed as a result of cell movements and interactions, each giving rise to a predictable lineage of tissue and organ derivatives. The morphogenetic rearrangement of cells establishes subpopulations, neighborhoods, and neighbors which interact and specialize as molecular signals are dispatched, thereby inducing adjacent cells, as well as the cells secreting them, to undergo divisions, engage in morphogenesis, and develop into tissues and organs. The markers for each of the embryonic germ layers, ectoderm, mesoderm and endoderm are respectively: β-Tubulin-III, Troponin, and Alpha-Fetoprotein.

Currently, there exists a widespread controversy over ESC research that emanates from the techniques used in their creation and usage. It would thus be advantageous to develop techniques of isolating stem cells that are as potent as ESCs, but do not require the destruction of a human embryo.

### SUMMARY

In one aspect of the present invention, there is provided an enriched preparation of Dermal Derived Human Stem Cells (DDhSCs) obtained from dermal fibroblasts that are capable of proliferation in vitro and differentiation to specialized tissue cell lineages. The DDhSCs are prepared by a method comprising: culturing a monolayer of dermal fibroblasts; inducing dedifferentiation of the dermal fibroblasts into DDhSCs; and collecting the DDhSCs that have detached from the monolayer. The DDhSCs are characterized in that the DDhSCs are positive for one or more of the stem cell markers β-tubulin III, troponin I, alpha-fetoprotein, E-cadherin, SSEA-1, SSEA-4, OCT ¾, SOX-2, CD-9, STRO-1, CD 105, Nanog, and PODXL.

In another aspect of the invention, a method is provided for propagating the DDhSCs. In one embodiment, a method is provided comprising culturing dermal fibroblasts on a monolayer of human fibroblasts, mouse embryonic fibroblasts, or a combination thereof; inducing dedifferentiation of the dermal fibroblasts into non-fibroblastic cells; transferring adherent non-fibroblastic cells to a collagen substrate; and culturing the non-fibroblastic cells for a period sufficient to promote the proliferation of morphologically undifferentiated DDhSCs.

In another aspect of the present embodiments, there is provided a method of inducing differentiation of DDhSCs in vitro comprising: obtaining undifferentiated DDhSCs; and providing a differentiating signal under conditions that induce unidirectional differentiation toward tissue cell lineages. Such partial or totally differentiated cell types include, but are not limited to, cellular lineages characteristic of the following tissues and organs: endocrine pancreas, exocrine pancreas, liver, lung, cartilage, bone, muscle, heart, and kidney.

In another aspect of the present embodiments, the DDhSCs may be used in the preparation of pharmaceutical compositions useful for organ and tissue regeneration.

In yet another aspect of the present embodiments, the DDhSCs are used in tissue engineering, regenerative medicine, or other cell-based therapy for the replacement or repair of body tissues that have been damaged by developmental defects, injury, disease, or the wear and tear of aging. The DDhSCs may be used alone or used in conjunction with any known biocompatible device, such as seeded into a matrix or scaffold for the purposes of tissue augmentation.

Other aspects of this invention would be evident for an expert in the field in view of the description of the invention.

### BRIEF DESCRIPTION OF THE DRAWINGS

FIG. 1. Micrograph at 50× magnification of colonies of DDhSCs.

FIG. 2. Micrograph at 200× magnification of colonies of DDhSCs.

FIG. 3A. Cloned Cells (DDhSC-003 on MEF). Micrograph at 200× magnification of a culture of DDhSCs cloned from one (1) cell on murine embryonic fibroblast feeder layers.

FIG. 3B. Cloned Cells (DDhSC-003 on MEF). Micrograph at 200× magnification of a 4 week culture of DDhSCs cloned from one (1) cell on murine embryonic fibroblast feeder layers.

FIG. 3C. Cloned Cells (DDhSC-003 on MEF). Micrograph at 200× magnification of a 4 week culture of DDhSCs cloned from 6 cells on murine embryonic fibroblast feeder layers.

FIG. 3D. Cloned Cells (DDhSC-003 on MEF). Micrograph at 200× magnification of a 4 week culture of DDhSCs cloned from 50 cells on murine embryonic fibroblast feeder layers.

FIG. 4A. Confocal fluorescent micrograph at 200× magnification of DDhSCs immunostained for Alpha-Fetoprotein, an endoderm marker gene.

FIG. 4B. Micrograph at 200× magnification without fluorescence showing the DDhSCs immunostained for Alpha-Fetoprotein, an endoderm marker gene.

FIG. 5A. Confocal fluorescent micrograph at 200× magnification of DDhSCs immunostained for Beta-Tubulin, an ectoderm marker gene.

FIG. 5B. Micrograph at 200× magnification without fluorescence showing the DDhSCs immunostained for Beta-Tubulin, an ectoderm marker gene.

FIG. 6A. Confocal fluorescent micrograph at 200× magnification of DDhSCs immunostained for Troponin, an ectoderm marker gene.

FIG. 6B. Micrograph at 200× magnification without fluorescence showing the DDhSCs immunostained for Troponin, an ectoderm marker gene.

FIG. 7A. Confocal fluorescent micrograph at 200× magnification of DDhSCs immunostained for PODXL, an ES cell marker gene.

FIG. 7B. Micrograph at 200× magnification without fluorescence showing the DDhSCs immunostained for PODXL, an ES cell marker gene.

FIG. 8A. Confocal fluorescent micrograph at 200× magnification of DDhSCs immunostained for CD-9, a progenitor cell marker gene.

FIG. 8B. Micrograph at 200× magnification without fluorescence showing the DDhSCs immunostained for CD-9, a progenitor cell marker gene.

FIG. 9A. Confocal fluorescent micrograph at 200× magnification of DDhSCs immunostained for Stro-1, a progenitor cell marker gene.

FIG. 9B. Micrograph at 200× magnification without fluorescence showing the DDhSCs immunostained for Stro-1, a progenitor cell marker gene.

FIG. 10A. Confocal fluorescent micrograph at 200× magnification of DDhSCs immunostained for Oct-¾, a progenitor cell marker gene.

FIG. 10B. Micrograph at 200× magnification without fluorescence showing the DDhSCs immunostained for Oct-¾, a progenitor cell marker gene.

FIG. 11A. Confocal fluorescent micrograph at 200× magnification of DDhSCs immunostained for SOX-2, a progenitor cell marker gene.

FIG. 11B. Micrograph at 200× magnification without fluorescence showing the DDhSCs immunostained for SOX-2, a progenitor cell marker gene.

FIG. 12A. Confocal fluorescent micrograph at 200× magnification of DDhSCs immunostained for SSEA-4, a progenitor cell marker gene.

FIG. 12B. Micrograph at 200× magnification without fluorescence showing the DDhSCs immunostained for SSEA-4, a progenitor cell marker gene.

FIGS. 13A and 13B. Dot plots showing FACS analysis of DDhSCs. The y axis shows staining with PE, the x axis shows staining with the negative controls IgG and anti-CD34-FITC conjugated antibodies.

FIGS. 14A-C. Dot plots showing FACS analysis of DDhSCs. The y axis shows staining with PE, the x axis shows staining with anti-CD105 and anti-E-Cadherin conjugated antibodies, compared to the IgG negative control.

FIGS. 15A-C. Dot plots showing FACS analysis of DDhSCs. The y axis shows staining with PE, the x axis shows staining with anti-CD105 and anti-E-Cadherin conjugated antibodies, compared to the IgG negative control.

FIGS. 16A-C. Dot plots showing FACS analysis of DDhSCs. The y axis shows staining with PE, the x axis shows staining with anti-Stro-1 and anti-SSEA-4 conjugated antibodies, compared to the IgG negative control.

FIGS. 17A and 17B. Dot plots showing FACS analysis of DDhSCs. The y axis shows staining with PE, the x axis shows staining with anti-CD-45 conjugated antibody, compared to the IgG negative control.

FIG. 18. Micrograph at 200× magnification of 16 weeks old fetal fibroblasts on collagen coated slides (2 days in culture).

FIG. 19A. Micrographs at 200× magnification of 20 weeks old fetal fibroblasts on collagen coated slides (2 days in culture).

FIG. 19B. Micrographs at 100× magnification of 20 weeks old fetal fibroblasts on collagen coated slides (2 days in culture).

FIG. 20. Micrograph at 200× magnification of 20 weeks old fetal fibroblasts on collagen coated slides (2 days in culture).

FIG. 21. Micrograph at 200× magnification of 24 weeks old fetal fibroblasts on collagen coated slides (2 days in culture).

FIGS. 22A and 22B. Micrographs at 100× and 300× magnification, respectively, of adult fibroblasts (DF204) on collagen coated slides (one week in culture).

FIG. 23. Micrograph of 24 weeks of adult fibroblasts (DF204) treated for 8 weeks with a signal-plex SP-41 (2 days in culture).

FIG. 24. Micrograph at 200× magnification of DDhSC differentiated into cartilage on a H-fiber scaffold (Histology of Cartilage, DDhSC -001 cells in H-fiber scaffold, 4mo, 200X).

FIG. 25. Micrographs showing cells stained with diphenylthiocarbazone (dithizone or DTZ), which stains zinc containing pancreatic β-cells crimson red.

FIG. 26. Micrographs showing dedifferentiated adult fibroblasts differentiated to Hepatocytes. A and B are stained with Anti human albumin antibody. C and D are phase contrast images.

FIG. 27. Micrographs showing cultured cells stained with Alizarin red S (A and B) for calcium, phase contrast image (A); mineral birefringence of the same aggregate viewed by polarized light microscopy (B), and also with Von Kossa stain (C and D).

FIG. 28. FACS analysis of the presence of CD34 antigens on the cultured cells before (A) and after (B) the hematopoietic cultures showing an increase in CD34 positive cells population in the cultures. C and D show the presence of CD34 positive cells by immunofluorescence study.

FIG. 29. Results of an immunofluorescence study. Cells were cultured two (2) weeks in neural proliferation medium and stained with anti-Musashi (A) and anti-Nestin (B). Cells cultured an additional two (2) weeks were stained with anti-β-tubulin III isoform to identify neurons (C), anti-Glial Fibrillary Acidic Protein to identify astrocytes (D), and anti-O1 to identify oligodendrocytes (E).

### DETAILED DESCRIPTION

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a, ” “an,” and “the” include plural reference unless the context clearly dictates otherwise. Thus, for example, a reference to “a cell” includes a plurality of such cells, as well as a single cell.

As used herein, “stem cells” refer to cells that can give rise to one or more cell lineages. Included are progenitor cells, totipotent cells, pluripotent cells, embryonic cells or post natal and adult cells. Also included are tissue-specific cells, including, but not limited to, cells committed to a particular lineage capable of undergoing terminal differentiation, cells that derive from tissue resident cells, and circulating cells that have homed to specific tissues.

As used herein, the terms “monolayer, ” “monolayer culture,” and “monolayer cell culture,” refer to cells that have adhered to a substrate and grow as a layer that is on average about one cell in thickness. Monolayers may be grown in any format, including but not limited to flasks, tubes, coverslips, wells of microtiter plates, roller bottles, etc. The terms “monolayer, ” “monolayer culture,” and “monolayer cell culture,” include layers of cells that have become “confluent,” wherein cells throughout a culture are in contact with each other creating what appears to be a continuous sheet of cells, and also include layers of cells that have not become confluent. When Dermal Derived Dedifferentiated human Stem Cells (DDhSCs) begin to form an aggregate, it often becomes a multilayered three dimensional structure that gradually lifts off the monolayer.

As used herein, the terms “medium, ” “media, ” “culture medium, ” “cell culture medium, ” “culture media,” and “cell culture media,” refers to media that are suitable to support the growth of cells in vitro (i.e., cell cultures). It is not intended that the term be limited to any particular culture medium. For example, it is intended that the definition encompass growth as well as maintenance media. Indeed, it is intended that the term encompass any culture medium suitable for the growth of the cell cultures of interest.

As used herein, the term “dermal fibroblast” refers to fibroblast cells or fibroblast-like cells of the dermis that possess the capacity to dedifferentiate to become Dermal Derived Human Stem Cells (DDhSC), which express the markers for pluripotency and resemble human embryonic stem cells (hESCs). The dermal fibroblast may be, for example, fetal fibroblasts (FF), neo-natal fibroblasts (NNF) or adult fibroblasts (AF) from the human dermis.

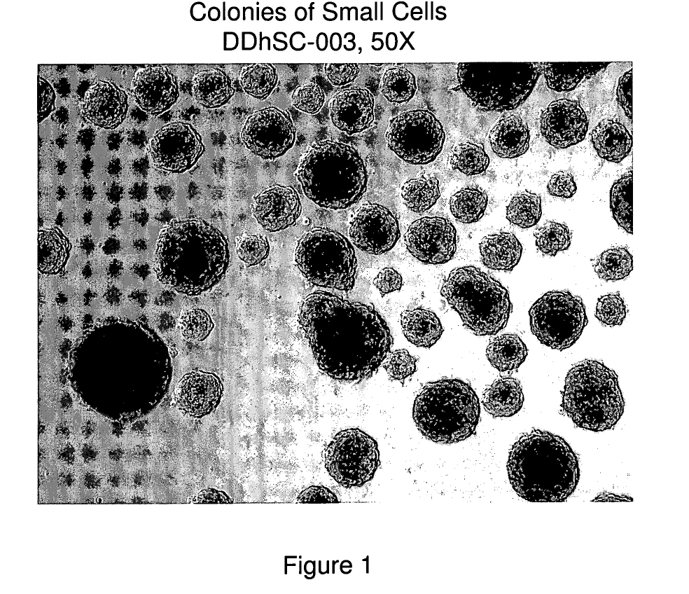

According to preferred embodiments, there is provided a method for isolating and dedifferentiating dermal fibroblasts inducing them to become pluripotent (the capacity to become one of a number of different cell types) and are useful in cell-based therapies and for regenerative medicine applications. The DDhSCs of the present embodiments may be predictably isolated from dermal fibroblasts and dedifferentiated such that they display characteristics similar to human embryonic stem cells derived from the inner cell mass of the blastula. FIGS. 1-3 show colonies of small cells that represent the DDhSCs of the present embodiments. These DDhSCs may then be made to differentiate into tissue cells having, for example, the features of endocrine or exocrine pancreas, liver, lung, kidney, heart, cartilage, bone or other cell types that have been induced, as shown by morphology, immunostaining, enzyme-linked immunoabsorbant assay, and reverse transcriptase-polymerase chain reaction analysis (See Dai et al., In vitro Cell Dev Biol Anim. 2002 April; 38(4):198-204).

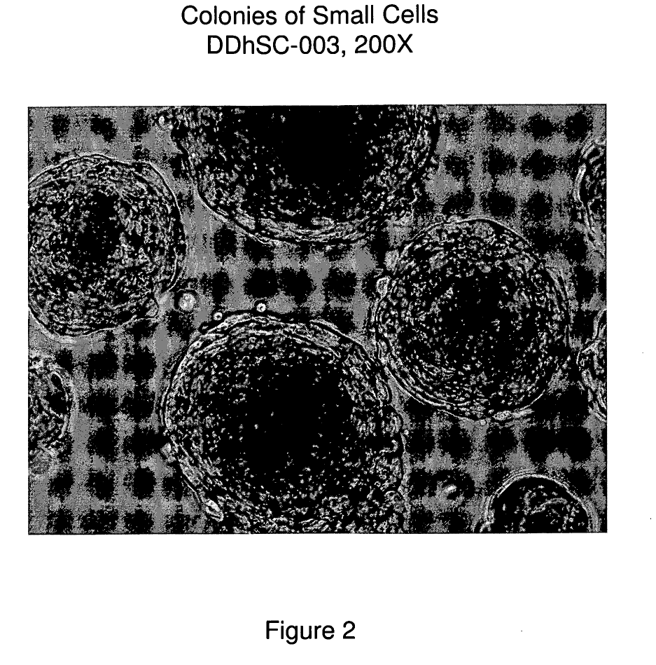

The DDhSCs may be derived from the dermal fibroblasts of humans of all ages, in addition to the period of gestation. Adult or fetal dermal fibroblasts may be cultured in vitro as a monolayer of cells to give rise to a subset of progenitor cells in the form of single cells and discrete colonies in the monolayer. FIGS. 1 and 2 shows colonies of DDhSCs, which consist of small round cells (DDhSCs) that remain in division and, in time, become spheres of cells that detach from the monolayer and float in the fluid medium above the monolayer of fibroblasts. When dissociated, the colonies consist only of non-fibroblastic small round cells, or DDhSCs.

The DDhSCs of the present embodiments are derived from dermal fibroblasts. Sources of dermal fibroblasts include, for example, skin of fetuses or skin taken postnatally at any age. DDhSCs may be derived from a subset of early gestational stages of human fetal dermal fibroblasts, and from adult human dermal fibroblast at various ages (the oldest tested was 93 years of age).

Any dermal fibroblast population is suitable and may be utilized to prepare the DDhSCs of the present embodiments. For example, normal adult human skin contains at least three distinct subpopulations of fibroblasts: papillary dermal fibroblasts, which reside in the superficial dermis; reticular fibroblasts, which reside in the deep dermis; and fibroblasts that are associated with hair follicles. See Sorrell et al., J Cell Sci. 2004 Feb. 15; 117(Pt 5):667-75. The DDhSCs of the present embodiments may be derived from any one of papillary dermal fibroblasts, reticular dermal fibroblasts, or dermal fibroblasts associated with hair follicles. Further, fibroblasts or fibroblast-like cells in parts of the body other than the dermis may also lend themselves to dedifferentiation and expression of the stem cell phenotype.

Although dermal fibroblast may be differentiated into certain cell types immediately after harvesting from the dermis, the fibroblast are preferably cultured in vitro under conditions that favor their dedifferentiation into the more highly potent DDhSCs. Thus, in one aspect of the present invention, there is provided a method of preparing DDhSCs involving a dedifferentiating or undifferentiating process. The method of dedifferentiating dermal fibroblast into DDhSCs includes: obtaining dermal fibroblasts from the dermis; culturing a population of dermal fibroblast under conditions to promote the proliferation of morphologically dedifferentiated DDhSCs; and recovering the dedifferentiated DDhSCs.

Dermal fibroblasts may be cultivated and dedifferentiated on: 1) a tissue culture substrate in a stem cell medium that favors the maintenance of stem cells in a undifferentiated or dedifferentiated condition; 2) on fibroblast feeder layers that support the DDhSCs growth and proliferation and inhibition of differentiation; 3) a combination of both 1 and 2; or 4) fibroblast monolayers exposed to Signal-plexes (see below). In a preferred embodiment, the tissue culture substrate is coated with an adhesive or other compound or substance that enhances cell adhesion the substrate (e.g., collagen, gelatin, or poly-lysine, etc.). Collagen-coated plates are most preferred. Where fibroblast feeder cells are utilized, mouse or human fibroblasts are preferably used; alone or in combination. It is preferred that the feeder cells are treated to arrest their growth, which may be accomplished by irradiation or by treatment with chemicals such as mitomycin C that arrests their growth. Most preferably, the fibroblast feeder cells are treated with mitomycin C. In preferred embodiments, the fibroblast feeder layer has a density of approximately 25,000 human and 70,000 mouse cells per cm², or 75,000 to 100,000 mouse cells per cm².

Preferably, the DDhSCs are cultured for a period of 4 to 24 days, and preferably for a period of 7 to 14 days. The DDhSCs, however, may be cultured for indefinitely long periods. For example, clones have been carried for greater than 4 months. Thus, the DDhSCs may be cultured for about 2 to about 4 months, about 4 to about 6 months, about 6 to about 8 months, about 8 to about 10 months, etc. Dedifferentiated DDhSCs detach from the monolayer and float in the medium, and, in this manner, may be identified. After a period of time, colonies of the dedifferentiated DDhSCs may be observed, which may be described as embryoid-like bodies or clusters of small, morphologically dedifferentiated cells that float in the medium.

The propagation of DDhSCs may be achieved using any known method. Preferably, the DDhSCs are grown on a fibroblast feeder layer, such as mitomycin treated MEF cells, for a period of about 4 to 14 days, and preferably from 7 to 10 days. Colonies of individual DDhSCs floating in the medium of feeder layer plates are removed, and the remaining attached cells are detached, for example by trypsinization, and transferred to tissue culture plates (e.g., collagen-coated plates) in a dedifferentiation medium. These DDhSCs are again cultivated for a period of 2 to 10 days, preferably for a period of 4 to 7 days, in a medium that encourages DDhSC colony formation in the monolayers of adult fibroblasts. In a preferred embodiment, the DDhSC growth medium comprises DMEM, 0.5% FBS, and the desired Signal-plex extract.

Any method known in the art for dedifferentiating cell cultures may be applied to the dermal fibroblast. Preferably, dermal fibroblasts are cultured in a medium containing various stem cell growth factors, or any other media known or designed to keep ESCs in an undifferentiated state. See e.g., Skottman et al., “Culture conditions for human embryonic stem cells.” Reproduction. 2006 November; 132(5):691-8; Amit et al., “Maintenance of human embryonic stem cells in animal serum- and feeder layer-free culture conditions,” Methods Mol Biol. 2006; 331:105-13; Yao et al., “Long-term self-renewal and directed differentiation of human embryonic stem cells in chemically defined conditions.” Proc Natl Acad Sci USA. 2006 May 2; 103(18):6907-12; Lu et al., “Defined culture conditions of human embryonic stem cells,” Proc Natl Acad Sci USA. 2006 Apr. 11; 103(15):5688-93; Amit et al., “Feeder layer- and serum-free culture of human embryonic stem cells,” Biol Reprod. 2004 March; 70(3):837-45; and U.S. Pat. No. 7,011,828, herein incorporated by reference in its entirety. For example, the dermal fibroblasts may be cultured using a base medium (e.g., IMDM, RPMI 1640, DMEM) supplemented with stem cell growth factors, antibiotics, and optionally with serum (e.g., fetal calf serum) or a serum substitute (e.g., Gibco BRL; may be used to avoid the possibility to viral or prion contamination) and/or other additives conventionally added to tissue culture media. Examples of stem cell growth factors include, but are not limited to, human multipotent stem cell factor, or embryonic stem cell renewal factor.

A preferred dedifferentiation medium comprises DMEM (GIBCO, without sodium pyruvate, with glucose 4500 mg/L) supplemented with about 5-20% FBS (HyClone, Utah), about 0.1 mM betamercaptoethanol, about 0.5-2% non-essential amino acids, about 05-2 mM glutamine, 0.5-2 mM penicillin, and 0.5-2 mM streptomycin.

Non-limiting examples of base media useful in the methods of the invention include Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's sale base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with non essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. A preferred medium for use in the present invention is DMEM. These and other useful media are available from GIBCO, Grand Island, N.Y., USA and Biological Industries, Bet HaEmek, Israel, among others. A number of these media are summarized in Methods in Enzymology, Volume LVIII, “Cell Culture”, pp. 62 72, edited by William B. Jakoby and Ira H. Pastan, published by Academic Press, Inc. Preferably, a high-quality basal media is used for the dedifferentiation of the dermal fibroblast.

Preferred supplements to the base medium are bovine serum albumin (BSA), Insulin, Transferrin, B-27, N-2, selenium, LDLs, PDGF, β-FGF, EGF, and mixtures and combinations thereof. The base medium may be serum-free or contain fetal serum of bovine or other species at a concentration of at least 1% to about 30%, preferably at least about 5% to 15%, and mostly preferably about 10%. Preferably, the fetal serum is heat inactivated. Embryonic extract of bovine, porcine, chicken, or other species may be present at a concentration of about 1% to 30%, preferably at least about 5% to 15%, most preferably about 10%.

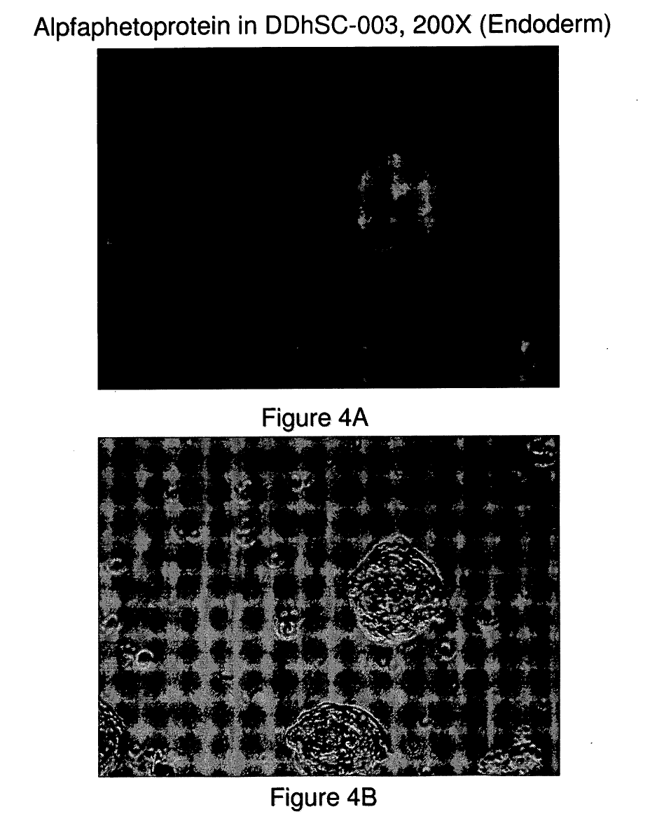

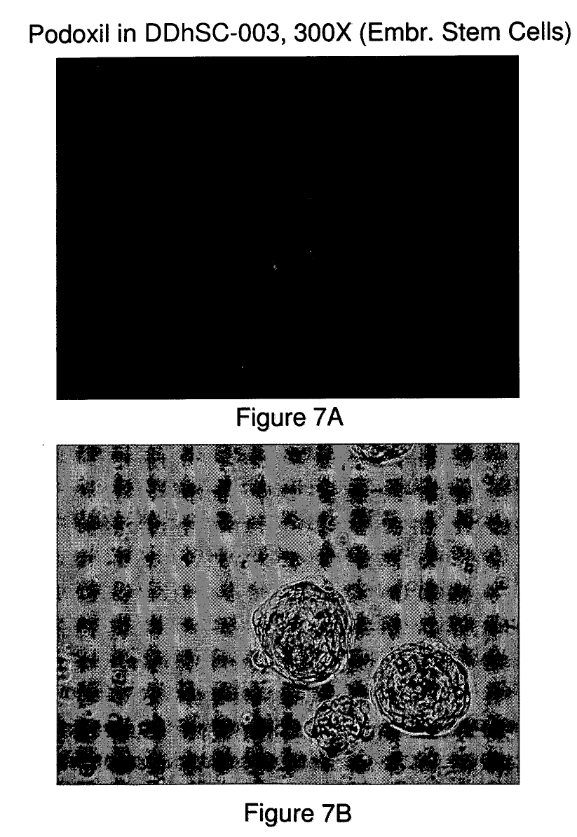

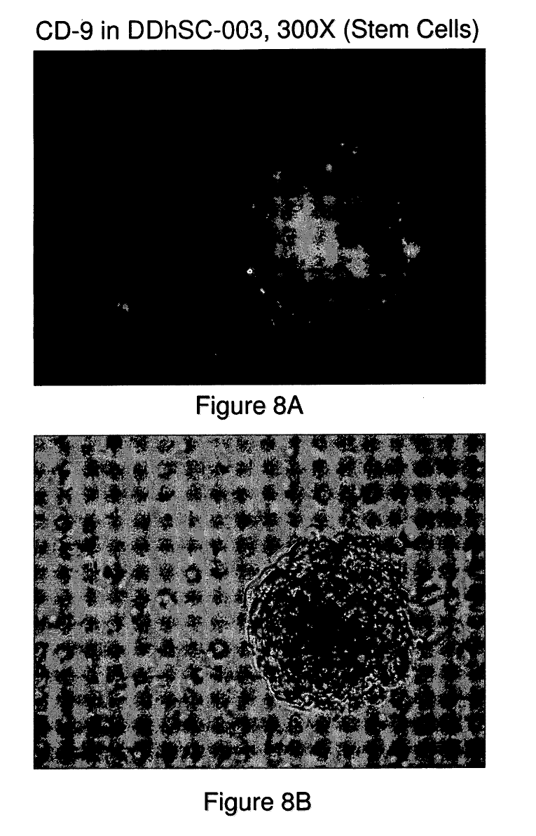

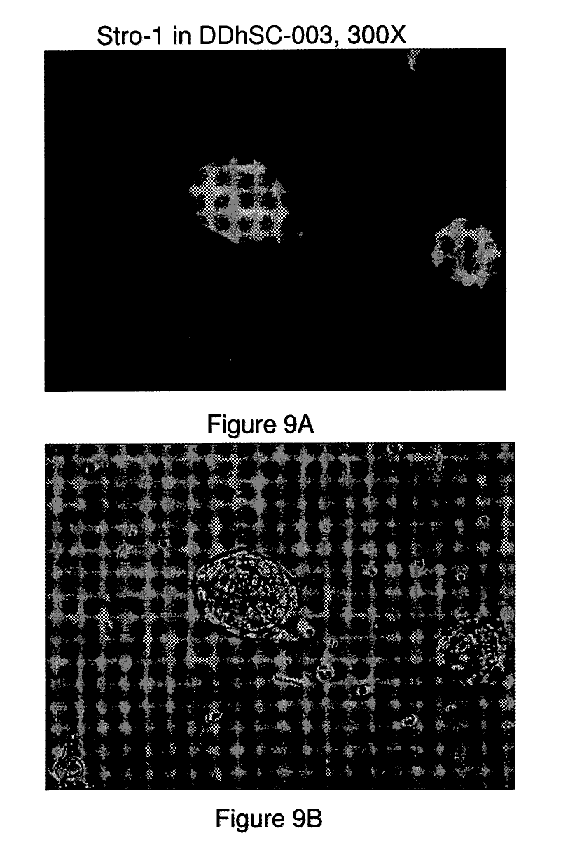

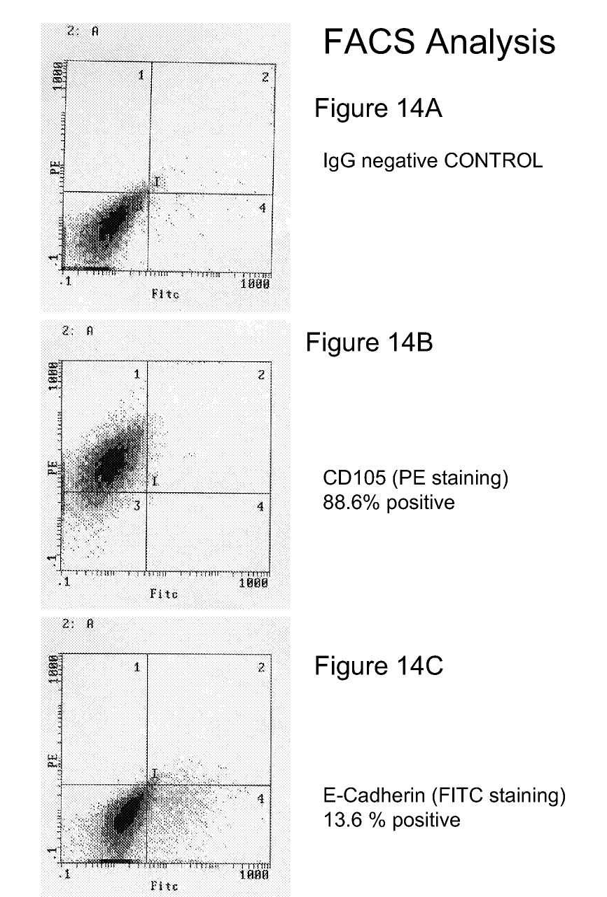

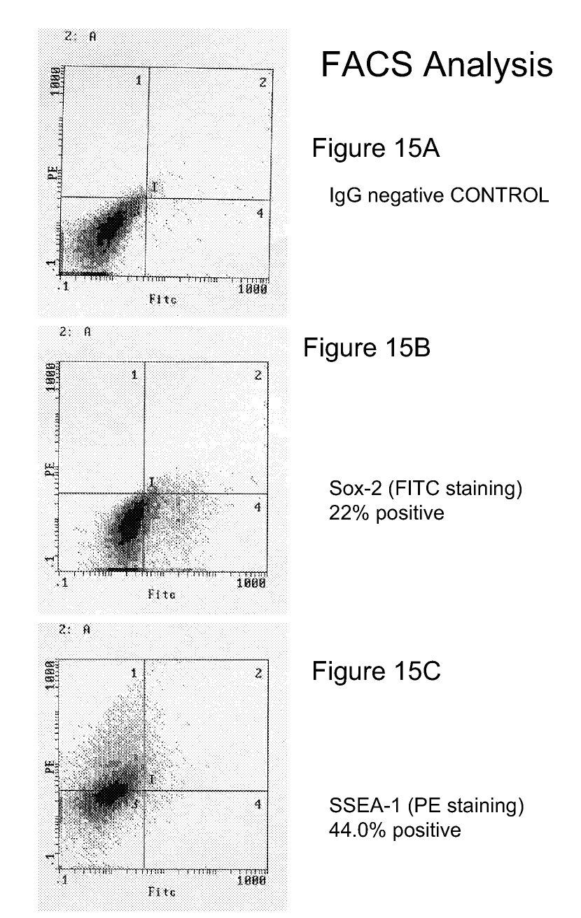

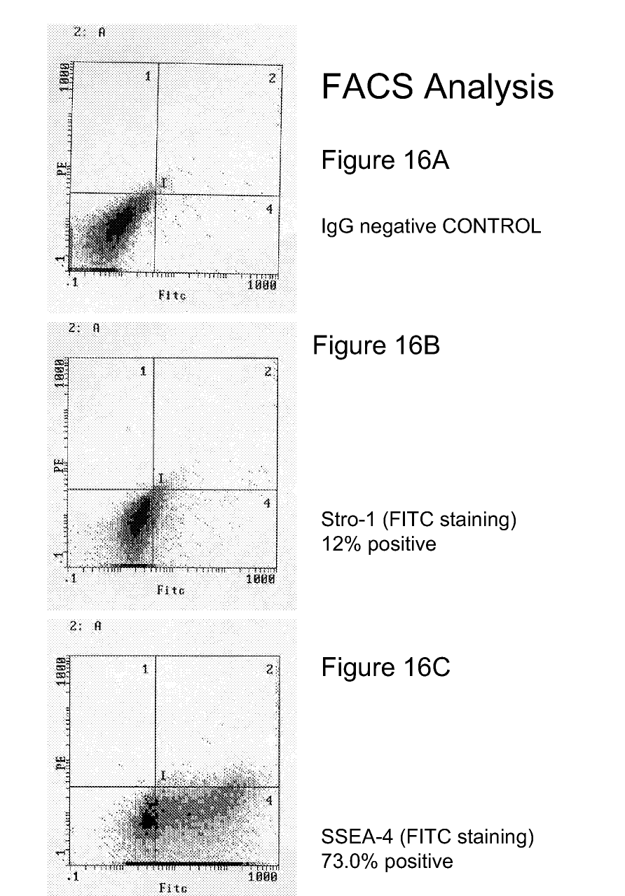

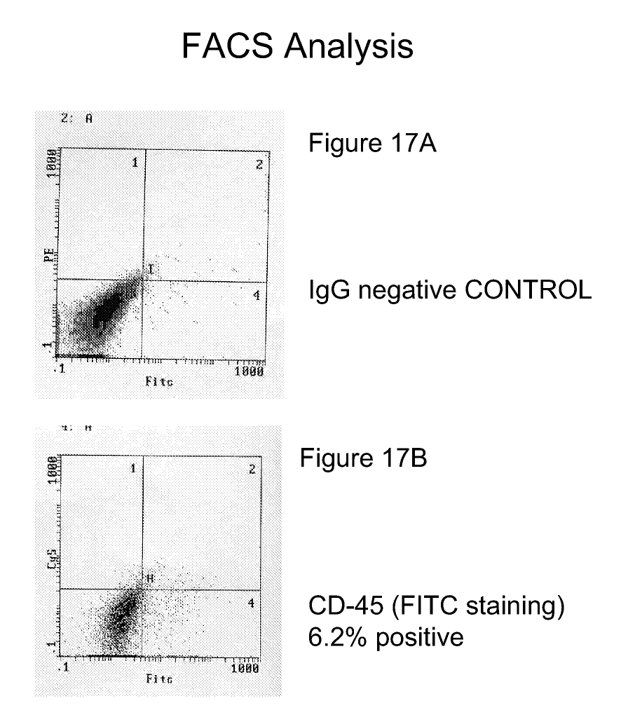

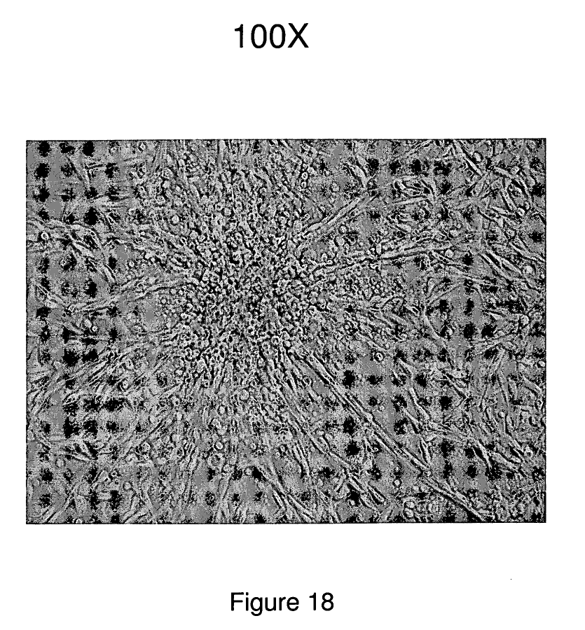

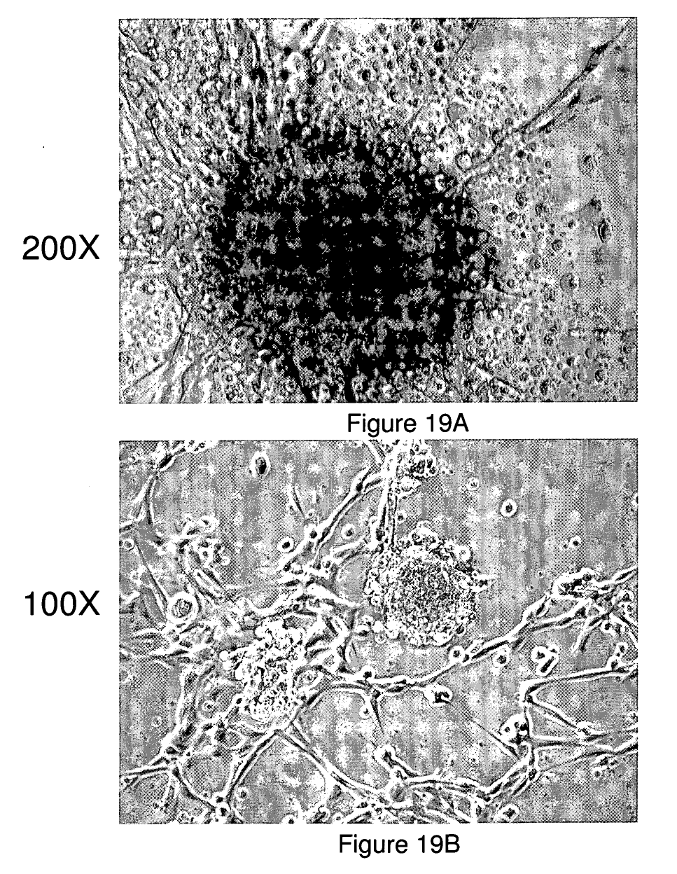

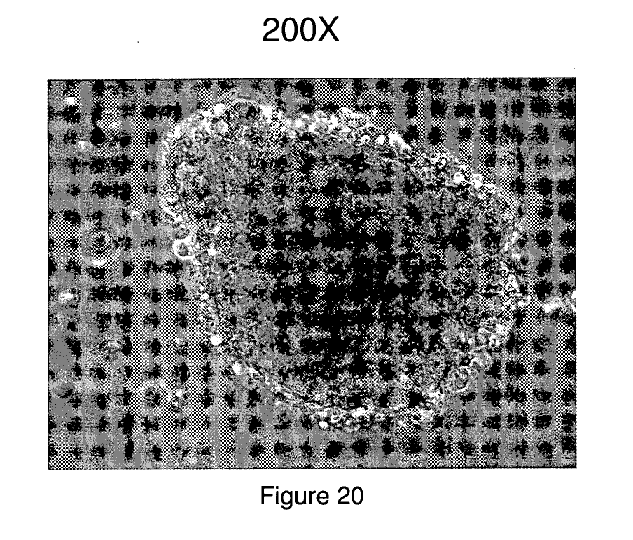

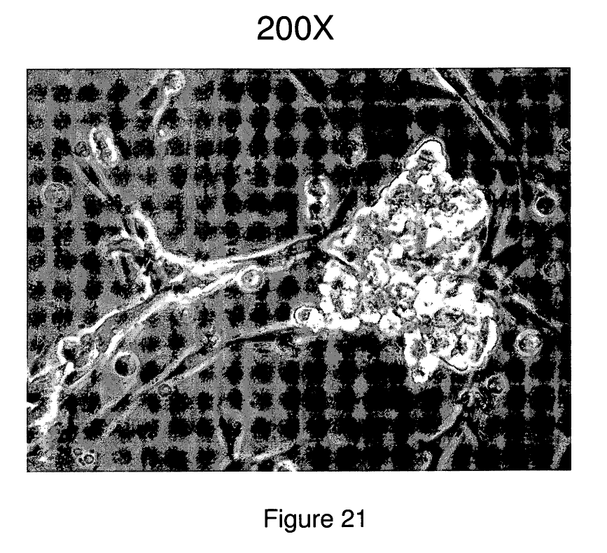

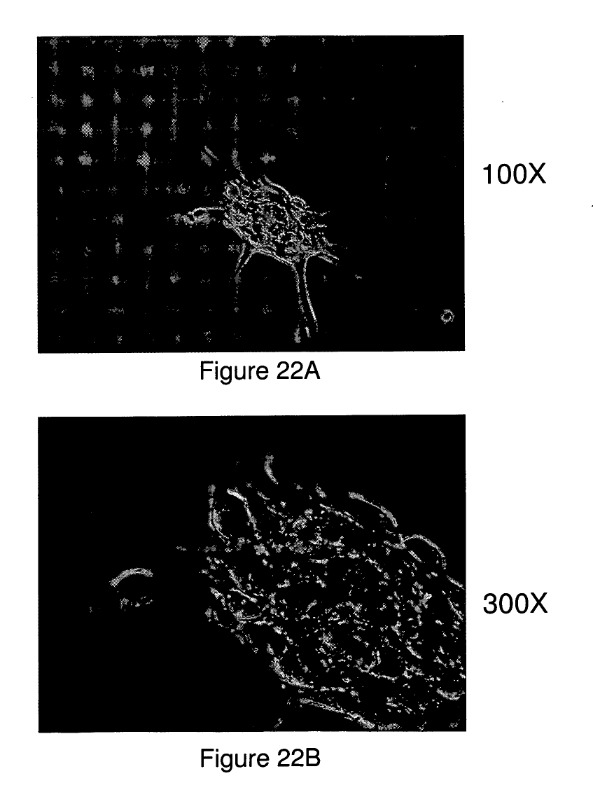

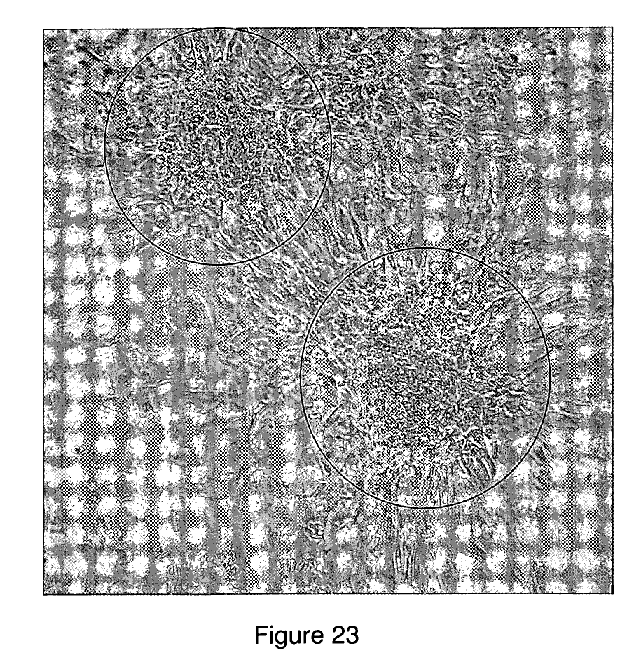

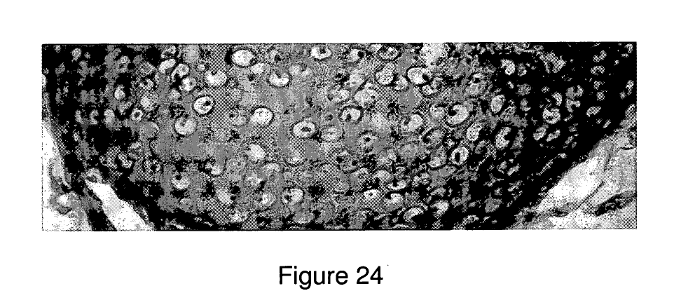

The DDhSCs of the present embodiments express markers of pluripotency, as well as markers of other stem cell properties. In this regard, the DDhSCs of the present embodiments resemble cells of the inner cell mass of the blastocyst from which the entire embryo and organism, except for the extracellular membranes, develop. The resemblance of DDhSCs to human ESCs is both morphological and functional. The DDhSCs exhibit both germ cell and progenitor cell markers (See FIGS. 4-17). Among the markers found are β-tubulin III, troponin I, alpha-fetoprotein, E-cadherin, SSEA-1, SSEA-4, OCT ¾, SOX-2, CD-9, TRA-1-60, TRA-1-81, CD105, Nanog, and PODXL. The DDhSCs are CD-45 and CD-34 negative, as measured by immunostaining and FACS analysis. Further, the size of the DDhSCs is on the order of magnitude of hESCs.

A population of cells such as dermal fibroblast that have been de-differentiated according to methods of the present invention into may be counted, sorted, and examined according their expression of one or more markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD105, Nanog, and PODXL. For example, DDhSCs of the present invention may be identified, counted, sorted, and examined according their expression of one or more markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD105, Nanog, and PODXL. According to some preferred embodiments, DDhSCs of the present invention may be identified, counted, sorted, and examined using flow cytometry methods (e.g., Fluorescence-activated cell sorting (FACS)).

According to some embodiments, methods are provided for making DDhSCs comprising the steps of a) culturing dermal fibroblasts on a monolayer of human fibroblasts, mouse embryonic fibroblasts, or collagen substrate; b) inducing dedifferentiation of the dermal fibroblasts into DDhSCs; and c) culturing the non-fibroblastic cells for a period sufficient to promote the proliferation of undifferentiated DDhSCs, characterized in that the DDhSCs are positive for one or more of the stem cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD 105, Nanog, and PODXL.

According to some embodiments, methods are provided for making DDhSCs comprising the steps of culturing dermal fibroblasts on a monolayer of human fibroblasts, mouse embryonic fibroblasts, or collagen substrate for a period sufficient to promote the proliferation of undifferentiated DDhSCs, characterized in that the DDhSCs are positive for one or more of the progenitor cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD105, Nanog, and PODXL.

According to some embodiments, the DDhSCs are positive for two or more of the stem cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD 105, Nanog, and PODXL.

According to some embodiments, the DDhSCs are positive for three or more of the stem cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD 105, Nanog, and PODXL.

According to some embodiments, the DDhSCs are positive for four or more of the stem cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD 105, Nanog, and PODXL.

According to some embodiments, the DDhSCs are positive for five or more of the stem cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD 105, Nanog, and PODXL.

According to some embodiments, the DDhSCs are positive for six or more of the stem cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD 105, Nanog, and PODXL.

According to some embodiments, the DDhSCs are positive for seven or more of the stem cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD 105, Nanog, and PODXL.

According to some embodiments, the DDhSCs are positive for seven or more of the stem cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD 105, Nanog, and PODXL.

According to some embodiments, the DDhSCs are positive for eight or more of the stem cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD 105, Nanog, and PODXL.

According to some embodiments, there is provided a composition comprising a population of dermal derived human stem cells produced by culturing dermal fibroblasts on a monolayer of human fibroblasts, mouse embryonic fibroblasts, or collagen substrate for a period sufficient to promote the proliferation of undifferentiated DDhSCs, characterized in that the DDhSCs are positive for one or more of the progenitor cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD 105, Nanog, and PODXL.

In response to appropriate differentiation signals, the DDhSCs differentiate along multiple pathways giving rise to many different phenotypes. According to another preferred embodiment, the DDhSCs are induced to differentiate in vitro to cells that express at least one characteristic of a specialized tissue cell lineage. The fetal, neonatal, and adult DDhSCs of the present embodiments may be induced to partially or totally differentiate into tissue cells having the features of tissue cells that include, but not limited to, endocrine pancreas, exocrine pancreas, liver, cartilage, bone, muscle, heart, and kidney.

The DDhSCs may be differentiated by placing the cells under the influence of signals designed to induce specifically the foregoing phenotypes. Any method of subjecting the DDhSCs to such signals may include, but not limited to, transfection of DDhSCs with genes known to cause differentiation, and/or exposing the DDhSCs to differentiation agents. For example, the DDhSCs may be genetically modified either stably or transitorily to express exogenous genes or to repress the expression of endogenous genes. In such a manner, the differentiation of the DDhSCs may be controlled. As an alternative example, the DDhSCs, and colonies thereof, may be induced to differentiate along a predictable pathway through the use of media that favors the maintenance in culture of a phenotype, such as that of the endocrine pancreas (See Lumelsky N. et al., “Differentiation of Embryonic Stem Cells to Insulin-Secreting Structures Similar to Pancreatic Islets,” Science 18 May 2001: 1389-1394). Methods of extracting growth and differentiation factors from fetal or neo-natal animal tissue are described in U.S. Pat. No. 6,696,074, entitled “Processing Fetal or Neo-Natal Tissue to Produce a Scaffold for Tissue Engineering,” herein incorporated by reference in its entirety.

In a preferred embodiment, nonadherent embryoid bodies and aggregates from supernatant of the dedifferentiated DDhSC cultures are collected and incubated them in commercially available lineage specific media. For example, for endoderm lineage, pancreatic medium may be used to induce differentiation of the cells into β-cells, insulin producing cells. For mesoderm lineage, osteogenic medium may be used to induce differentiation of the cells into osteoblasts. Also hematopoietic lineage into CD34 positive cells. For ectoderm lineage, neural proliferation medium may be used to proliferate the neural stem cells followed by induction to differentiation by neural differentiation medium.

In a preferred embodiment, a method is provided for signaling DDhSCs to differentiate by bringing them into contact with complexes of signaling molecules (called “Signal-plexes” or “S-p”) prepared from embryonic, fetal, or post-natal animal tissue have been used to induce stem cells to express the same tissue and organ phenotypes as those from which the Signal-plexes were derived. Signal-plexes are prepared by a method that involves harvesting, lysing, homogenizing and filtering animal tissue to remove solids to form extracts. Signal-plexes are cell free extracts, and methods of making and using thereof, are described in U.S. Patent Publication No. 2002/0146401, entitled “Generation and Use of Signal-plexes to Develop Specific Cell Types, Tissues and/or Organs,” herein incorporated by reference in its entirety.

Additionally, each of the tissue specific signaling complexes has been found to be responsible for inducing the dedifferentiation of cultured human fetal dermal fibroblasts to the DDhSC, and then induce the differentiation of the DDhSCs to become many different cell types, e.g., bone cells, cartilage cells, insulin secreting cells, glucagon secreting cells, and chymotrypsin secreting cells. Thus, according to another preferred embodiment, dermal fibroblast may be cultured in the presence of tissue specific signaling complexes derived from developing tissue, or Signal-plexes, to induce their dedifferentiation to DDhSCs and redifferentiation into specialized tissue cells.

Sources of Signal-plexes include, but are not limited to, the developing tissues of mammals such as pigs, sheep, and cows. The cell-free molecular signals derived from the developing mammalian tissues are capable of inducing adult dermal fibroblasts to dedifferentiate into DDhSCs and are then able inducing the DDhSCs to express a differentiated lineage, which is the same cellular/tissue lineage from which the Signal-Plex was derived. Using tissue specific signaling complexes, experimental results have shown that mouse embryonic stem cells can be predictably induced, for example, to express the liver phenotype that produces serum albumin, cardiac myocytes that form a beating tissue (heart) that persists for months, and calcifying bone cells that make a hardened mass.

The DDhSCs of the present embodiments may provide an important resource for rebuilding or augmenting damaged tissues, and thus represent a new source of medically useful progenitor cells. In a preferred embodiment, the DDhSCs may be used in tissue engineering and regenerative medicine for the replacement of body parts that have been damaged by developmental defects, injury, disease, or the wear and tear of aging. The DDhSCs provide a unique system in which the cells can be differentiated to give rise to specific lineages of the same individual or genotypes. The DDhSCs therefore provide significant advantages for individualized cell therapy. For example it would be possible to use them for fabricating skin. TEI's EBM collagen matrix, disclosed in U.S. Pat. No. 6,696,074, incorporated herein by reference, is now used in many thousands of patients to repair dural tissue, rotator cuff, and other tissues, and could serve as a dermal matrix when seeded with undifferentiated small round DDhSCs that are then released from their uncommitted status by incubating them in a suitable medium, such as that previously reported in Bell et al., Proc. Natl. Acad. Sci. 76: 1274- 1278 (1979). In addition, components of the stem cell medium, such as β-FGF, may be withheld.

In accordance with a preferred embodiment, any known matrix or scaffold may be seeded with DDhSCs or differentiated DDhSCs. For example, TEI's Collagen H-fiber foam, disclosed in U.S. Pat. No. 5,709,934, incorporated herein in its entirety, may be used as a matrix that can be seeded with DDhSCs that are then induced to differentiate and form a tissue of a specific phenotype(s). The cell seeded neo-dermis structure, whether it be EB Matrix, Collagen II-fiber foam, or other scaffold product, can be overlaid with Matrigel™, a solubilized basement membrane preparation (BD Bioscience), incorporated in a collagen solution of a concentration 0.5 mgs/ml at 20 degrees C. Alternatively, a thin collagen gel alone, or supplemented as necessary with other factors, may be used as the support layer for the epidermis. To initiate gelling of the collagen, the neo-dermis may be incubated at 37° C. in a CO2 incubator. When the mixture has gelled (after 1.0 hours) a suspension (10⁵ cells/cm²) of small round DDhSCs may be plated onto the gel surface and incubated for 48 hours with the skin equivalent immersed in the medium, such as the medium disclosed in Bell et al., Science, 211:152-154 (1981). After 2-5 days the skin-equivalent may be air lifted and the Ca⁺⁺ concentration of the medium is increased from 0.02 mmol/L to 1.88 mmol/L so that the developing epidermis can begin to keratinize. Air lifting consists of raising the skin equivalent out of the medium to a level that exposes its surface to the atmosphere within the CO2 incubator. Several alternative methods are well known in the art, such as the method disclosed in Aberdam, D Int. J. Dev. Biol., 48:203-206 (2004). The feasibility of making an allogeneic living skin equivalent has been proved by Bell and colleagues (See Sher et al., Transplantation, 35: 552-557 (1983)), which is produced and marketed by Organogenesis, Inc. The DDhSCs may be allografted after they are induced to differentiate along a specific pathway.

The following examples are illustrative, but not limiting, of the methods and compositions of the present invention. Other suitable modifications and adaptations of the variety of conditions and parameters normally encountered in therapy and that are obvious to those skilled in the art are within the spirit and scope of the embodiments.

### Example 1

Fetal fibroblasts (FF), neo-natal fibroblasts (NNF), and adult fibroblasts (AF) from the human dermis were dedifferentiated to become dermally derived progenitor cells that resemble hESCs. Samples of skin from fetuses of 8, 10, 12, 14, 16, 18, 20 and 24 weeks of gestational age were obtained from Advanced Bioscience Resources, Alameda, Calif. 94501. Samples of foreskins from new-born boys were obtained from the Boston Medical Center (Boston University) Boston Mass. 02118. Skin biopsies from adults between 19 and 93 years of age were obtained from the National Disease Research Interchange, 8 Penn Center, 8th Floor, at 1628 JFK Boulevard, Philadelphia Pa. 19103, and from Metro West Dermatology, Framingham, Mass. 01702. By request, samples of skin of all ages were sent in DMEM without serum. All suppliers tested the tissues that were sent, and found them to be free of the pathogens HIV and hepatitis B.

Two methods (Meth-1 and Meth-2) were used for preparing FF, NNF and AF for dedifferentiation. Dermal fibroblasts were isolated in the same way for both methods. Namely, when skins arrive, they are cut to a size of about 5×5 mm, they are washed twice in PBS, rinsed for 15-20 seconds in 70% ethanol, and the skin pieces are washed twice in PBS before being spun down and transferred to a solution of 0.05% trypsin, plus 2 mM EDTA for 16 hours at 4° C. to free the fibroblasts attached to the dermal matrix. Soybean trypsin inhibitor was used at 40 U/ml to stop the digestion. After refrigerating the tube, the trypsin/EDTA was aspirated off, the dermal pieces were washed with serum free DMEM, and then transferred into 10 ml of fresh DMEM in a 15 ml tube. The dermal pieces were vortexed for 10 seconds twice and the contents (i.e., fluid plus cells plus dermal matrix plus epidermal remnants) were pipetted on to a 40 μ cell strainer. The extracellular materials of the dermal pieces and other fragments were collected on the strainer, while the fibroblasts passed through the filter. The filter was rinsed with serum-free DMEM to flush cells through it. We washed cells with PBS and determined their viability and the cell number using trypan blue. For Meth- 1, cells were not passaged before dedifferentiation. For Meth 2, cells were passaged before dedifferentiation. Using Meth-2, after a gentle spin, the dermal fibroblasts were resuspended in 10.0 ml of DMEM (GIBCO)+10% FBS. Cells of each strain and age were then plated at 5×10³ cells in 75 cm² tissue culture flasks. After 1-3 passages, 10⁶ cells were frozen in a total volume of 1.5 ml made up of DMEM with 20% FBS and 10% dimethylsulfoxide (DMSO) in 2 ml vials at −80°.

To compare feeder layer culturing, and alternatives to using a feeder layer, one third of the dermal fibroblasts were routinely plated on 6 well plates (Costar) each with a feeder layer consisting of mitomycin treated murine embryonic fibroblast cells (MEF) purchased from ATCC (VA, USA), or more recently mitomycin treated 8 week old fetal human fibroblasts tested for division potential and their resistance to dedifferentiation. The second third of the dermal fibroblasts were plated on 6 well plates (Costar) coated with collagen (100 ug/ml). The last third of the dermal fibroblasts were plated on collagen coated 12 mm diameter cover glasses. Two types of media were preferred (see below). M-I was used for cells on feeder layers, while M-II was used for cells plated on collagen. A minimum of three strains each of FF, NNF, and AF were dedifferentiated by Meth-I. Alternative culture methods for adult cells were based on the use of SignalPlexes, discussed previously and in Example 8 below.

For these experiments, two types of media were used for inducing dedifferentiation and for maintaining dedifferentiated dermal fibroblasts in the non-differentiated condition, M-I and M-II. M-I was the medium used for fibroblasts plated on feeder layer, which comprised knock-out (KO) DMEM (GIBCO), 15% KO serum replacement, (GIBCO), 1.0% non-essential amino acids (NEAA), 0.1 mM mercaptoethanol (Sigma), 1.0% penicillin (GIBCO), 1.0% glutamine (GIBCO) and 6 ng/ml βFGF (R&D Systems). M-II was used for fibroblasts plated on collagen coated plates or on coverglasses. It comprised embryonic stem cell basal medium (StemCell Technologies, Vancouver NC Canada) with 0.5 mg/ml insulin, 5 mg/ml transferrin, 0.52 μg/ml sodium selenite, 1× N-2 supplement (Stem Cell Tech.), 1× B-27 supplement (StemCell Tech.), 2.5% bovine serum albumin and β-FGF at 6 ng/ml (R&D Systems).

Dedifferentiation was carried out with strains prepared by Meth-I and Meth-II. For Meth-II, dermal fibroblasts were passaged, frozen down, and, when needed, thawed for use, washed 3 times in DMEM with no FBS before dedifferentiation.

### Example 2

A total of fifteen strains were studied. Eight strains of FF, three strains of NNF, and four strains of AF were dedifferentiated to the small cell phenotype. The oldest donor was 93 years of age. Colonies of small round cells were observed forming at various loci among the plated monolayers of fibroblasts cultured in either M-1 on feeder layer, or in M-2 on collagen coated cover glasses. Few colonies formed in M-2 on collagen coated plastic. AF, FF and NNF, cultured in vitro as monolayers under conditions favoring maintenance of the hESC phenotype, underwent dedifferentiation and became small cells that formed colonies in the monolayer. It was not determined whether all colonies are actually clones, as from time to time, the apparent movement of fibroblasts into the zone of colony formation was observed. The colonies grow in size three dimensionally and bud-off from the monolayer. When dissociated, they consist only of small round cells. Colonies of small round cells can remain in division and in time become larger sphere-shaped colonies of cells that detach from the monolayer and reside in the fluid medium above the monolayer of fibroblasts until they are removed.

Using a panel of antibody markers, it was determined that the cells stain for expression of pluripotency, and other stem cell properties, and, that the small cells are similar, including size, to human embryonic stem cells (hESC) derived from the inner cell mass of the blastula. Further, it was determined that an entire population of small cells dedifferentiated from adult fibroblasts could be redifferentiated to the fibroblast phenotype in less than a day by returning them to the basic DMEM medium with 10% FBS, 1.0% penicillin and 1.0% glutamine.

Using SignalPlexes, the formation of colonies of small round fetal cells, or Dermal Derived Dedifferentiated human Stem Cells (DDhSCs), were found to be formed after periods of 6 to 14 weeks in 3d fiber foam scaffolds, and sometimes after longer periods in culture, as compared with the current rapid appearance of aggregates (about 10 days) in cultures of FF, NNF, or AF dermal fibroblasts. Alternative culture methods for adult cells may be based on the use of SignalPlexes, discussed previously and in Example 8 below.

### Example 3

### FF, NNF, and AF Dermal Fibroblasts on Collagen Coated Plates

Dermal fibroblasts from Example 1 were plated at 1.5×10⁶ cells/well on collagen (100ug/ml) coated 6 well plates in embryonic stem cell basal medium (Stem Cell Tech., Canada) containing insulin, transferrin, selenium, N-2 supplement, B-27 supplement, 2.5% Bovine serum albumin, and β-FGF at 6 ng/ml. Colonies of small cells (FIG. 1) were observed after 10 days. After 16 days, nonadherent aggregates were pooled from 2 wells and trypsinized (0.25%) in test tubes for 15 min at 37° C. Cells may then again be plated on collagen coated 12 well plates using the stem cell basal medium. Colonies formed in cultures of fetal, neonatal, and adult DDhSCs are seen to detach from the monolayer and to float in the medium.

### Example 4

### Fetal Dermal Fibroblasts on Feeder Layers

Fetal dermal fibroblasts from Example 1 were plated at 2×10⁶ cells/well on mitomycin treated murine embryonic fibroblasts (MEF) feeder layer or on mitomycin treated adult fibroblast in wells of a four well plate. The medium used was knock out DMEM (high glucose) supplemented with 10% heat inactivated FBS, 0.1 mM beta-mercapto ethanol, 1% non-essential amino acids, 1 mM glutamine, 1 mM penicillin/streptomycin, and β-FGF.

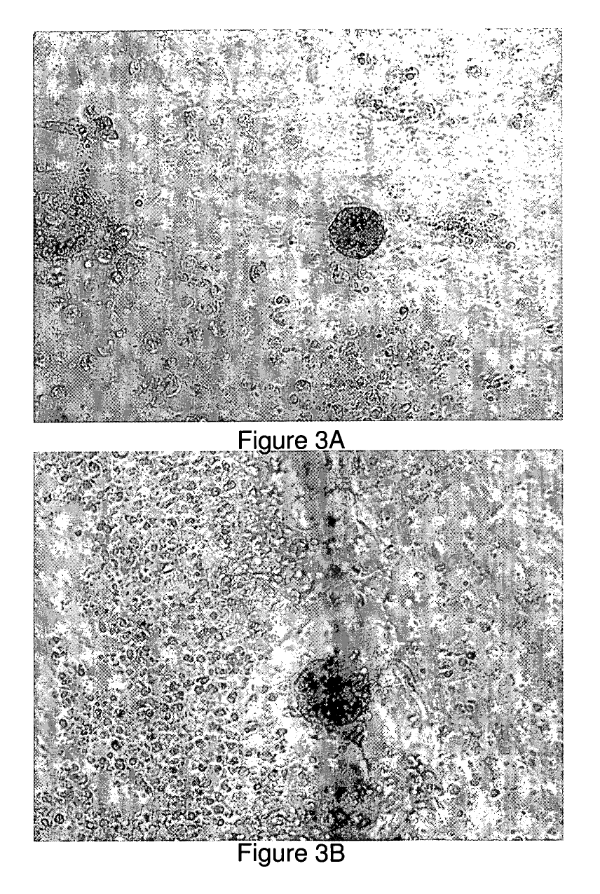

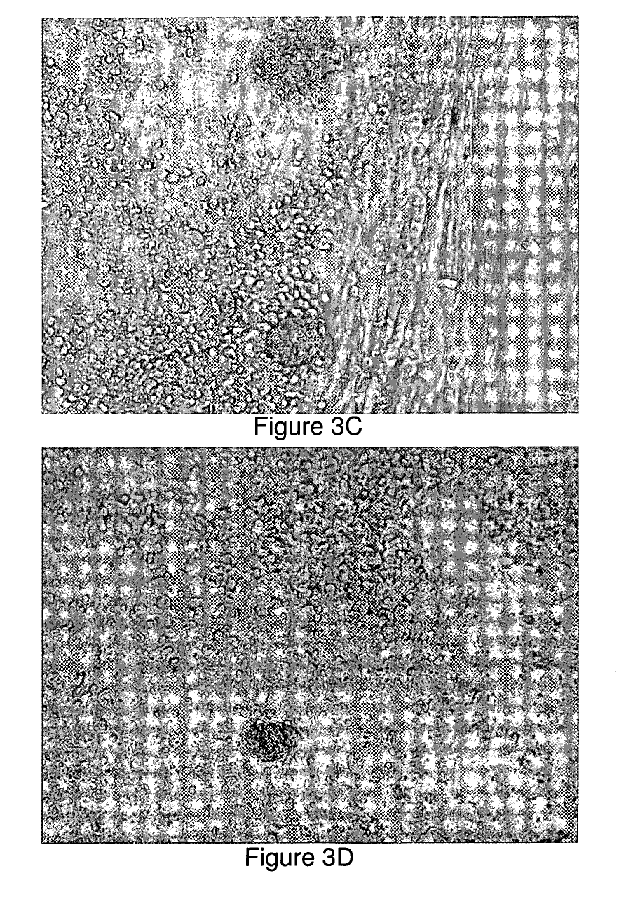

Dermal Fibroblasts (such as the DDhSC strain DDhSC 003 shown in FIGS. 1-3) were grown on mitomycin treated MEF. After one week cells were trypsinized (0.25 for 7 min at 37°) and plated on collagen coated 100 mm glass plates using embryonic stem cell basal medium as above. Four days later, a large number of colonies of small round cells were seen floating in the dish. Some of the colonies were dissociated, as described above, and the cells were cloned in 24 or 96 well plates at serial dilutions down to 1 cell per well (FIGS. 3A-3D). At dilutions of about 1.0 cell per/well, 6, 12, 25 50 and 100 cells/well, thousands of cells in 96 well plates were seen at a magnification of 400× in most fields looked at by 30 days.

### Example 5

### Method for Accelerating and Inducing Formation of Large Numbers of Floating Colonies Made up of DDhSCs.

After growing dermal fibroblasts on a feeder layer of mitomycin treated MEF cells for 7 days, few colonies have formed, while the rate of appearance of colonies from dermal fibroblasts grown on collagen is as described above was of the order of about 10-20. The colonies of Dermal Derived Dedifferentiated human Stem Cells (DDhSCs) floating in the medium of feeder layer plates are removed, and the remaining attached cells are detached by trypsinization and transferred to collagen coated plates in a stem cell medium containing β-FGF. Hundreds of colonies of small round cells are seen by 4 days in each well of a six well collagen coated plate.

### Example 6

### Flow Cytometry Analysis

Flow cytometry analysis was conducted on three strains of Dermal Derived Dedifferentiated human Stem Cells (DDhSCs): DDhSC 001; DDhSC 002; and

DDhSC 003). Each was obtained and propagated from a different donor. The results showed that each of the strains expressed markers for each of the three germ layers: β-Tubulin-III, Troponin- 1, and Alpha-Fetoprotein.

For flow cytometry analysis, non-adherent colonies were collected from the DDhSCs cultures. Colonies were dispersed into constituent small cells using trypsin (0.25%) for 10 min at 37°C. After washing, cells were counted, and stained with fluorochrome-conjugated antibodies to surface antigens and in some cases for intracellular proteins. Antibodies used for surface phenotype determination included anti-CD34, anti-CD45, anti-CD105 (Qbend10, Immunotech, Westbrook, Me.), and anti E-Cadherin (R & D Systems, MO) antibodies. Cells were stained in the presence of staining buffer (PBS with 2% fetal bovine serum). After staining, the cells were fixed with 4% formalin (Sigma, St. Louis, Mo.) for 1 hour at room temperature. For intracellular proteins, cells were fixed overnight with 4% formalin and permeabilized with ice cold acetone (Sigma) and antibodies to stage specific embryonic antigen- I (SSEA-1), stage specific embryonic antigen-4 (SSEA-4), anti-PODXL, anti-Sox-2 (R & D systems, Minneapolis, MN) were used following the staining protocol of the manufacturer. Stroma cells antibody Stro-1 (Zymed, San Francisco, Calif.) was also used to test for the presence of stroma positive cells in colonies.

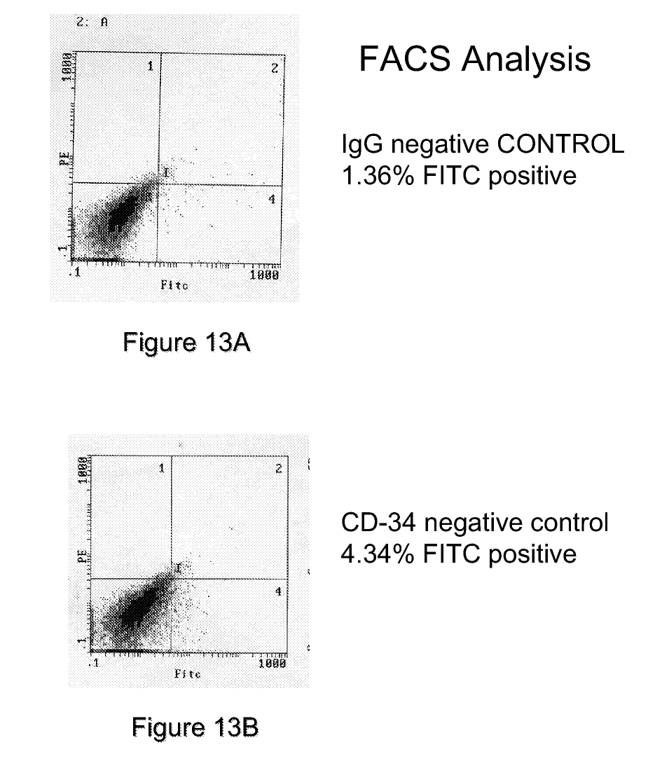

Flow cytometry was performed using a FACSCalibur flow cytometer (Becton Dickinson). Appropriate controls included matched isotype antibodies to establish positive and negative quadrants; as well as appropriate single color stains to establish compensation. For each sample, at least 5,000 list mode events were collected. E-cadherin, SSEA-1, SSEA-4, OCT ¾, SOX-2, STRO-1, CD105 and PODXL were all positive. CD45 and CD-34 were negative. Results of the FACS analysis on the DDhSC 003 strain are provided in FIGS. 13-17 and in Table 1 below.

TABLE 1
FACS Analysis of DDhSC

| | |
|---|---|
| E-cadherin | 13.6% |
| SSEA-1 | 44.0% |
| SSEA-4 | 73.0% |
| OCT ¾ | 18.85% |
| SOX-2 | 22.0% |
| STRO-1 | 12.0% |
| CD105 | 88.6% |
| PODXL | 22.20% |
| CD-34 | 4.3%* |
| CD-45 | 6.2%* |
| TRA-1-60 | 16% |
| TRA-1-81 | 12% |

Numbers indicate the percentage of cells stained by the indicated MAb as determined by FACS.
*Indicates negative result.

### Example 7

### Immunofluorescence Staining

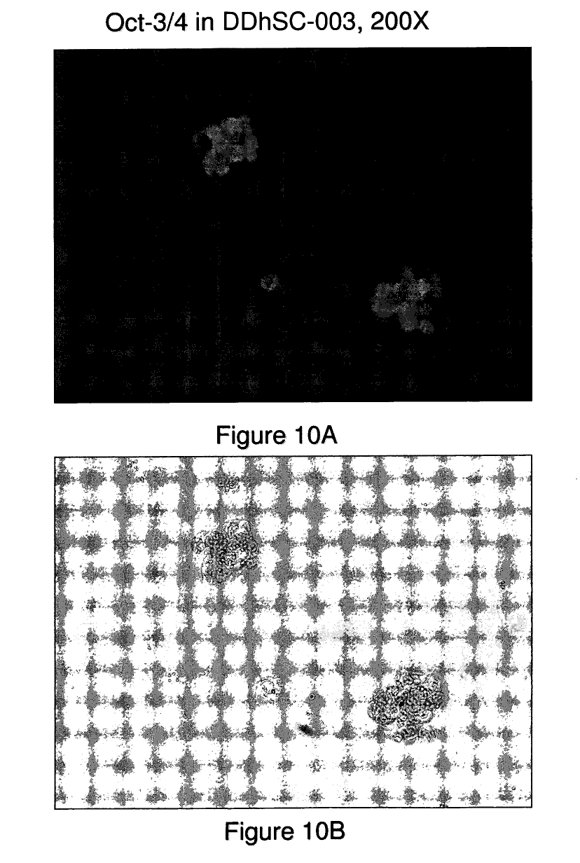

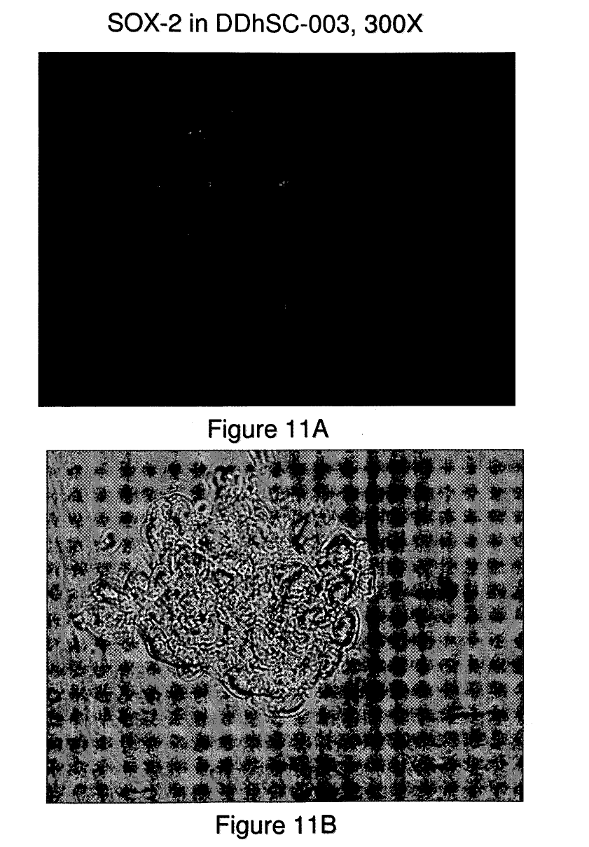

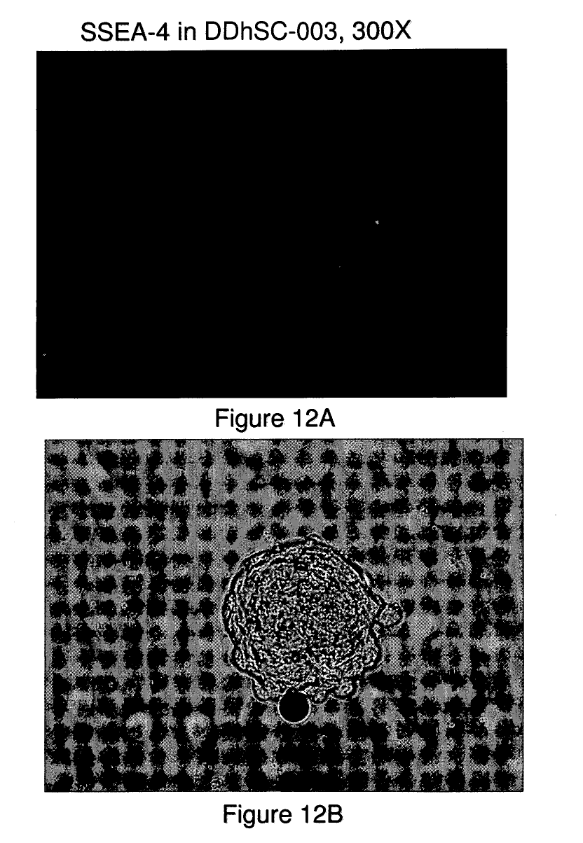

Cells dedifferentiated from the dermis were also tested for the expression of each of the three germ layer markers by Immunofluorescence Staining. DDhSCs cells were fixed in 4% Formalin in PBS for 15 min at room temperature. The cells were rinsed with PBS containing 2% BSA (Sigma) and permeabilized with ice cold Acetone for 15 min. After washing thrice with PBS-BSA, the cells were incubated with primary antibodies to octamer biding transcription factor (Oct) ¾ (FIGS. 10A and 10B), Sox-2 (FIGS. 11A and 11B), SSEA-1, SSEA-4 (FIGS. 12A and 12B), CD9, TRA-1-60, TRA-1-81, anti-Nanog antibodies (R & D systems, Minneapolis, Minn.) for 1 hour at 4° C. Cells were washed and incubated with secondary antibody of FITC-conjugated goat anti-mouse IgG (1:100; Santa Cruz Laboratories, CA) for 30 min at 4° C.

Following three washes with PBS, the cells were examined with an Axiovert-100 fluorescence microscope (Carl Zeiss, NY) equipped with a Micro Color Moticam 300C Digital camera. SSEA-1, SSEA-4, SOX-2, CD-9 and Oct ¾ and PODXL were positive.

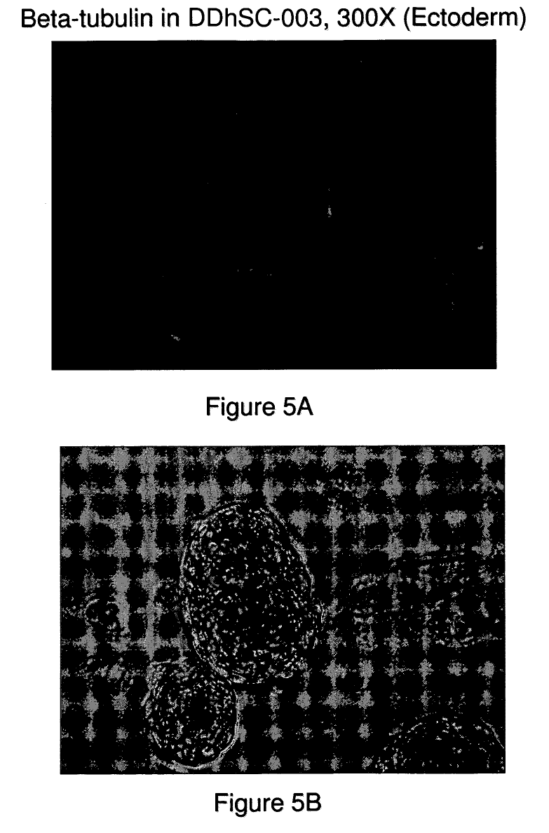

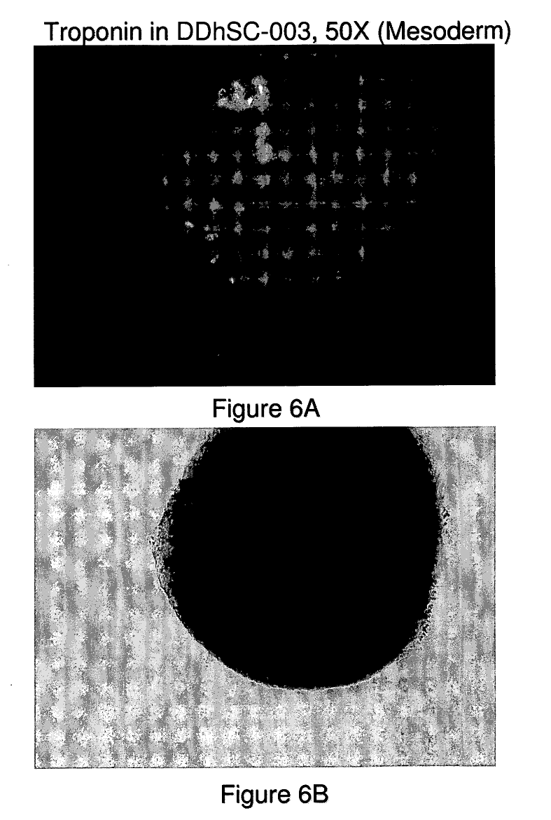

Antibodies against proteins specific to cells of each germ layer and expressed by hESC were used to examine the dermal derived dedifferentiated cells as follows: (1) for ectoderm the expression of β-tubulin III (FIGS. 5A and 5B), a neuron specific molecule; (2) for mesoderm cardiac troponin I (FIGS. 6A and 6B); and (3) for endoderm alpha-fetoprotein (FIGS. 4A and 4B). All were strongly positive, indicating that the DDhSCs give rise to embryoid-like bodies. Primary antibodies to β-tubulin III, and alpha-fetoprotein were purchased from Sigma and cardiac troponin I was purchased from Santa Cruz Laboratories. The results showed that each of the strains expressed markers for each of the three germ layers: Tubulin-III, Troponin, and Alpha-Fetoprotein.

### Example 8

### Preparation of Tissue Specific Signals or “SignalPlexes”

SignalPlexes, disclosed in U.S. Patent Publication No. 2002/0146401, incorporated herein by reference in its entirety, were prepared from fetal pigs of two ages 40 and 80 days of gestation obtained from Johnsonville Sausage LLC based in Chicago Ill.

Developing tissues and organs are processed sterilely under cold room conditions. Samples of each type of tissue taken are cut into pieces smaller than 5×5 mm after placing them in 50 ml or 250 ml tubes with caps and weighed. HBSS (Hanks Balanced Salt Solution) added to each tube in an amount of 3 mls/gram of tissue. Also added are Aprotenin 10 ug/ml, EDTA 2 mM, and Polymethylsulphafluoride 0.1 mM; all are final concentrations. A 22 mm assembly of a Tissue Tearer Homogenizer was used for samples weighing less than 20 grams. The closed blade was run at 30 k rpm for 5 min to reduce small samples to a fine consistency. For tissue samples weighing more than 20 grams a Waring Laboratory blender was used. Following homogenization all tubes were weighed and balanced tubes were transferred to a rocker/roller in a cold room for 30 min to promote further extraction. Next, in a Jouan centrifuge, tubes were spun at 4000 RPM for 30 min at 2° C. With a pipette, the supernatants were transferred to 30 ml Nalgene tubes and spun at 40,000g at 4° C. in a Beckman JA25 centrifuge. The pH of the supernatants was adjusted to ˜7±0.2 if needed. To sterilize the supernatant, it is filtered through a 0.2 μ filter for sterilization as detailed in U.S. Pat. No. 6,696,074 to Dai et al., the entire contents of which are incorporated by reference.

### Example 9

### Preparation of Human Cartilage from Human Fetal Dermal Cells In Vitro

Fetal skin at 8 weeks of age is collected, cut into small pieces and treated with trypsin at 4° C. for 16 hours. The cells are resuspended in medium containing 10% FBS in DMEM. The cells in suspension are decanted with the supernatant and plated on to culture plates to establish a primary culture of the fibroblastic skin cells, as described in Example 1. The cells are seeded into a collagen foam scaffold in three dimensions before the addition of cartilage-specific SignalPlex (S-p). Suitable collagen scaffolds are described in U.S. Pat. No. 6,696,074, entitled “Processing Fetal or Neo-Natal Tissue to Produce a Scaffold for Tissue Engineering,” and in U.S. Pat. No. 5,800,537, entitled “Method and Construct for Producing Graft Tissue from an Extracellular Matrix,” both of which are incorporated herein by reference in their entireties.

Cell free DNAse treated cartilage S-p is prepared from 80 day developing porcine cartilage, as described Example 8. The total extract is spun at 4000 RPM for 30 minutes at 4° C., passed through two layers of 1.0 mm pore size cheese cloth and then through a 0.2 μm syringe filter before adding 30 μg of signaling complex (prepared from 80 day fetal porcine developing cartilage) to 1 ml of culture medium now containing 0.5% FBS. Medium is changed every three to four days with the addition of fresh cartilage S-p. In samples that receive the cartilage-specific signaling complex, cartilage forms in vitro in approximately three months. In controls that have not received the signaling complex, no cartilage forms by five months.

### Example 10

### Trans-differentiation of Stem Cells from Dedifferentiated Adult Human Skin Cells into Different Lineages: Insulin producing cells & Hepatocytes (Endoderm)

Nonadherent embryoid bodies and aggregates were collected from supernatant of the dedifferentiated DDhSC cultures and incubated them in commercially available lineage specific media. For Endoderm lineage, pancreatic medium was used to induce differentiation of the cells into β-cells, insulin producing cells.

A) Insulin producing cells: Aggregates, embryoid bodies and non adherent cells of DD cells of a 93 year old donor incubated in M-II using Meth-II were collected and cultured in pancreatic proliferating medium. After two weeks in the pancreatic proliferating medium, the medium was changed to differentiating medium as the manufacturer instructs. After 4 weeks, two assays were carried out. The first uses diphenylthiocarbazone (dithizone or DTZ), which will stain zinc containing pancreatic β-cells crimson red. The second assay is for insulin production in which cell supernatants were collected and ELISA assays were conducted using an Insulin Kit supplied by Alpco Diagnostics.

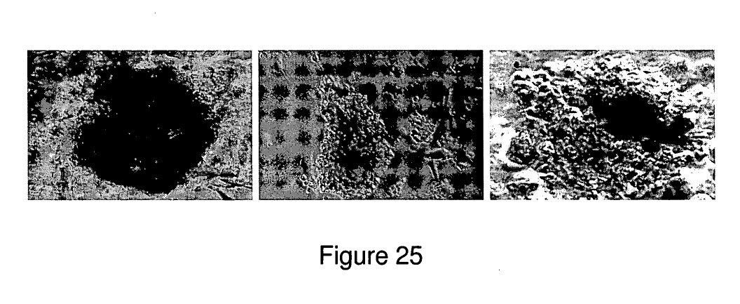

To stain zinc containing β-cells, the cells were stained with 100 ug/ml of diphenylthiocarbazone (dithizone or DTZ) after 2 weeks of culture in pancreatic differentiating medium. Cells in the culture dishes were incubated at 37° C. for 15 minutes in the DTZ solution. After, the dishes were rinsed three times with HBSS. Clusters stained crimson red were examined microscopically. FIG. 25 shows cells stained with diphenylthiocarbazone (dithizone or DTZ).

The measurement of insulin production of the cultured DDhSC cells was carried out using an Insulin Kit supplied by Alpco Diagnostic. Briefly cells were washed twice with Krebs-Ringer Bicarbonate (KRB) buffer and incubated for two hours in fresh buffer supplemented with 25 mM glucose. Supernatant was collected and ELISA assays for insulin were carried out as the manufacturer directed. The data below represents insulin content of respective media.

| Supernatant | Insulin per U/L |
|---|---|
| Supernatant from differentiated DD cells | 200.0 U/L |
| Krebs Buffer | 0.008 U/L |
| 0.008 U/L | |
| Medium for inducing differentiation of insulin | 25.0 U/L |
| producing cells | |

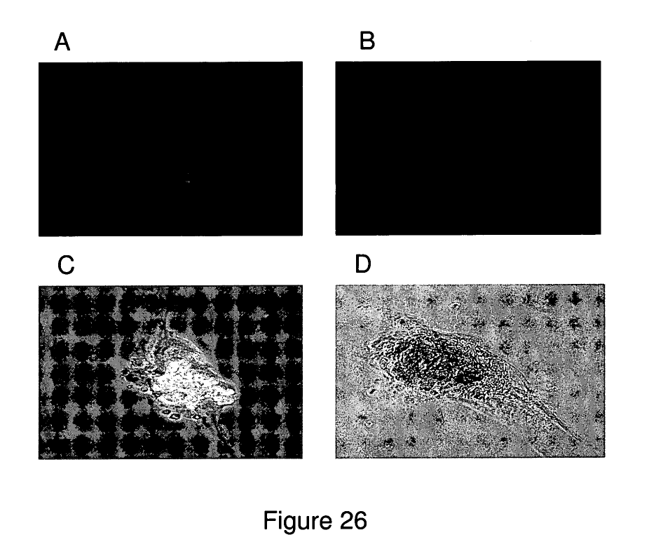

B) Hepatocytes: Dedifferentiated cells were cultured in commercially available hepatocyte medium and growth factors for 3 weeks. Cells were stained with anti human albumin antibody as shown in FIG. 26.

### Example 11

### Trans-differentiation of Stem Cells from Dedifferentiated Adult Human Skin Cells into Different Lineages: Osteogenic & Hematopoietic Cells (Mesodermal) Phenotype

Nonadherent embryoid bodies and aggregates were collected from supernatant of the dedifferentiated DDhSC cultures and incubated them in commercially available lineage specific media. For Mesoderm lineage, osteogenic medium was used to induce differentiation of the cells into osteoblasts. Also hematopoietic cell lineage into CD34 positive cells.

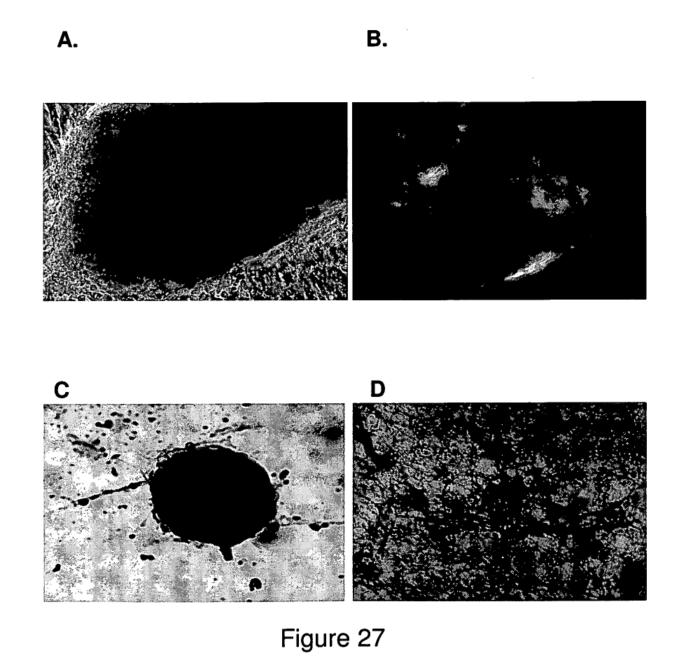

A) Osteogenic Cells: Dedifferentiated DDhSC cells were induced to trans-differentiate to an osteogenic (mesodermal) phenotype by a three week exposure to a fortified osteogenic medium. Cultured cells were stained with Alizarin red S for calcium, phase contrast image (FIG. 27 A); mineral birefringence of the same aggregate viewed by polarized light microscopy (FIG. 27 B), and also with Von Kossa stain (FIG. 27 C &D). The presence of calcium in the extracellular matrix surrounding the differentiating stem cell derivatives was demonstrated by staining with Alizarin red S (A) and by polarization microscopy which revealed its crystalline character (B).

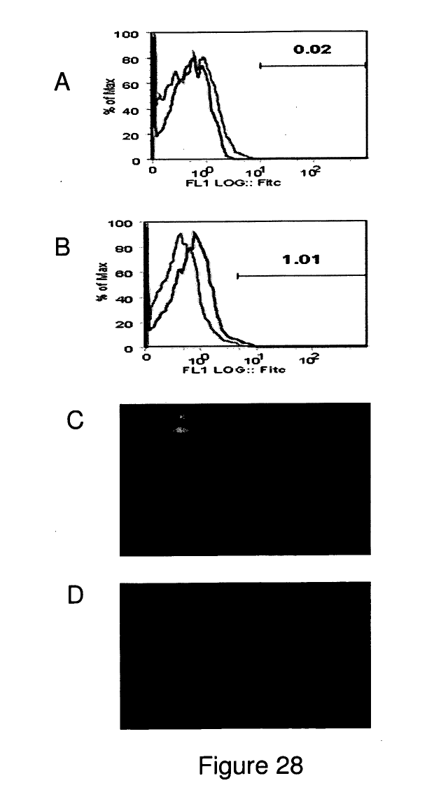

B) Hematopoietic Cells: Dedifferentiated DDhSC cells were cultured in hematopoietic media supplemented with BMP-4, VEGF, SCF, FLK-2/Flt-3 ligand, EPO, TPO, G-CSF growth factors for 4 days. Non-adherent cells were collected after 4 days and stain with antibody to CD34 antigen to assess the expression of CD34 antigens. FIGS. 28A and B shows the FACS analysis of the presence of CD34 antigens on the cultured cells before (A) and after (B) the hematopoietic cultures. There is an increase in CD34 positive cells population in the cultures. FIGS. 28C and D shows the presence of CD34 positive cells by immunofluorescence study.

### Example 12

### Differentiation of Stem Cells from Dedifferentiated Adult Human Skin Cells into Different Lineages: Neural (Ectodermal) Phenotypes

Nonadherent embryoid bodies and aggregates were collected from supernatant of the dedifferentiated DDhSC cultures and incubated them in commercially available lineage specific media. For Ectoderm lineage, neural proliferation medium was used to proliferate the neural stem cells followed by induction to differentiation by neural differentiation medium.

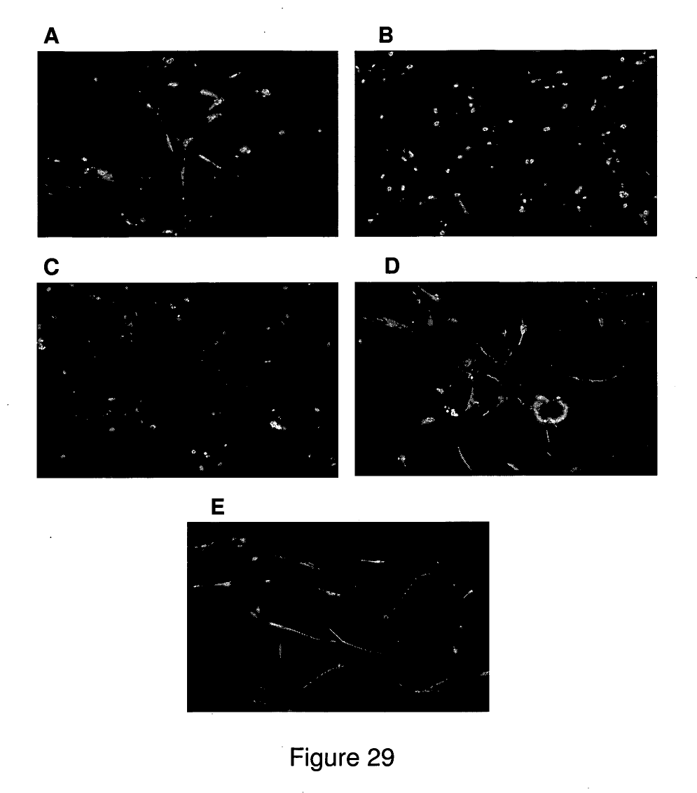

Floating embryoid bodies, aggregates and non adherent cells were collected, dissociated and re-suspended in neural stem cell proliferation medium in 24 well plates and 2 chamber slides coated with laminin and poly-1-lysine. After 2 weeks the medium of some wells were changed to neural differentiation medium and the cells were incubated for another 2 weeks. Immunofluorescence study was done to analyze the phenotypes of trans-differentiated cells. We used anti-Musashi and anti-Nestin to recognize neural progenitors in the cells cultured in neural proliferating medium that can give rise to neurons, glia and oligodendrocytes. For immunofluorescence study with differentiated cells, anti-β-tubulin III isoform was used to identify neurons, anti-Glial Fibrillary Acidic Protein (GFAP) was used to identify astrocytes, and anti-O1 to find oligodendrocytes. Results of these studies are shown in FIG. 29. After two weeks in neural proliferation medium, cells express Musashi-1 (FIG. 29A) and Nestin (FIG. 29B). Cells cultured two weeks in neural proliferation medium followed by another two weeks in neural differentiation medium express β-tubulin III (FIG. 29C), GFAP (FIG. 29D), and Oligodentrocyte O-1 (FIG. 29E).

All patent applications, patents, texts, and literature references cited in this specification are hereby incorporated herein by reference in their entirety to more fully describe the state of the art to which the present invention pertains.

As various changes can be made in the above methods and compositions without departing from the scope and spirit of the invention as described, it is intended that all subject matter contained in the above description, shown in the accompanying drawings, or defined in the appended claims be interpreted as illustrative, and not in a limiting sense.

What is claimed is:

1. A method of making Dermal Derived Human Stem Cells (DDhSCs) comprising the steps of:
a. culturing dermal fibroblasts on a monolayer of human fibroblasts, mouse embryonic fibroblasts, or collagen substrate;
b. inducing dedifferentiation of the dermal fibroblasts into DDhSCs; and
c. culturing the non-fibroblastic cells for a period sufficient to promote the proliferation of undifferentiated DDhSCs, characterized in that the DDhSCs are positive for one or more of the stem cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD 105, Nanog, and PODXL.

2. The method of claim 1, further comprising identifying, counting, sorting, or examining DDhSCs according to their expression of one or more markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD105, Nanog, and PODXL.

3. The method of claim 1, wherein the DDhSCs are cultured for a period sufficient to promote the formation of DDhSC cell clusters or colonies.

4. The method of claim 1, whereby the dedifferentiated cells express levels of telomerase activity consistent with the condition of immortality.

5. The method of claim 1, wherein the dermal fibroblasts are cultured using a medium supplemented with β-FGF and albumin.

6. The method of claim 1, wherein the dermal fibroblasts are cultured using a culture medium supplemented with fetal serum and β-FGF.

7. The method of claim 1, wherein the dermal fibroblasts are cultured using a culture medium supplemented with serum substitute and β-FGF.

8. The method according to claim 1, wherein the fibroblast feeder cells are arrested in their growth.

9. The method of claim 1, wherein the dermal fibroblasts are cultured in a medium comprising fetal serum and a tissue extract obtained from an embryonic, fetal, or postnatal tissue.

10. The method of claim 9, wherein the tissue extracts are produced by
a. harvesting animal tissue;
b. lysing and homogenizing the tissue to produce extracts;
c. filtering the tissue extracts such that the extracts are substantially free of cell membranes, nuclear membranes, nuclei, mitochondria, and microorganisms; or
d. extracts derived from DDhSCs or DDhSCs redifferentiated to any cell or tissue type of the body.

11. The method of claim 10, wherein the tissue extracts are obtained from endocrine pancreas, exocrine pancreas, liver, lung, cartilage, bone, muscle, heart, or kidney.

12. The method of claim 1, further comprising the step of propagating individual DDhSCs within or on feeder layers as a means of perpetuating strains of DDhSCs, wherein the feeder layers comprise human fibroblasts, mouse embryonic fibroblasts, a collagen substrate, or a combination thereof.

13. The method of claim 1, wherein the DDhSCs are phenotypically undifferentiated.

14. An isolated Dermal Derived Human Stem Cell (DDhSC) obtained from the method of claim 1.

15. A method of inducing differentiation the DDhSCs of claim 1 comprising
a. obtaining DDhSCs; and
b. culturing the DDhSCs under conditions which favor their differentiation to specialized tissue cells.

16. The method of claim 15, wherein the DDhSCs are cultured in a lineage specific media.

17. The method of claim 16, wherein the lineage specific media is endoderm lineage specific media, mesoderm lineage specific media, or ectoderm lineage specific media.

18. The method of claim 15, wherein the DDhSCs are cultured in a medium comprising fetal serum and a tissue extract obtained from an embryonic, fetal, or postnatal tissue.

19. The method of claim 18, wherein the tissue extracts are produced by
a. harvesting animal tissue;
b. lysing and homogenizing the tissue to produce extracts; and
c. filtering the tissue extracts such that the extracts are substantially free of cell membranes, nuclear membranes, nuclei, mitochondria, and microorganisms.

20. The method of claim 19, wherein the tissue extracts are obtained from endocrine pancreas, exocrine pancreas, liver, lung, cartilage, bone, muscle, heart, or kidney.

21. A pharmaceutical composition that includes a cell population according to claim 1 and an acceptable pharmaceutical vehicle.

22. A pharmaceutical composition according to claim 21 wherein the cells and, optionally, the additional components, are included in a three-dimensional biocompatible synthetic or biologic matrix.

23. A pharmaceutical composition according to claim 22 wherein said three-dimensional biocompatible synthetic or biologic matrix is of a microparticle, microsphere, nanoparticle, or nanosphere type.

24. A pharmaceutical composition comprising a dedifferentiated, programmable cell of dermal fibroblast origin, wherein said dedifferentiated, programmable cell of dermal fibroblast origin expresses β-tubulin III, troponin I, alpha-fetoprotein, E-cadherin, SSEA-1, SSEA-4, OCT ¾, SOX-2, CD-9, TRA-1-60, TRA-1-81, CD105, Nanog, and PODXL.

25. A method of transplanting Dermal Derived Human Stem Cells (DDhSCs) into a host, said method comprising:
a. obtaining dermal fibroblasts;
b. inducing dedifferentiation of the dermal fibroblasts into DDhSCs, whereby the DDhSCs are positive for β-tubulin III, troponin I, alpha-fetoprotein, E-cadherin, SSEA-1, SSEA-4, OCT ¾, SOX-2, CD-9, CD 105, Nanog, and PODXL, and
c. implanting the DDhSCs into a host.

26. The method of claim 25, wherein the dermal fibroblast are obtained from the host.

27. A composition comprising a population of dermal derived human stem cells produced by culturing dermal fibroblasts on a monolayer of human fibroblasts, mouse embryonic fibroblasts, or collagen substrate for a period sufficient to promote the proliferation of undifferentiated DDhSCs, characterized in that the DDhSCs are positive for one or more of the stem cell markers selected from the groups consisting of β-tubulin III, troponin I, alpha-fetoprotein, E-cadherin, SSEA-1, SSEA-4, OCT ¾, SOX-2, CD-9, TRA-1-60, TRA-1-81, CD 105, Nanog, and PODXL.

28. Use of an isolated cell population according to claim 1 to prepare a pharmaceutical composition for the repair and augmentation of bodily tissue selected from the group consisting of cartilage, bone, muscle, heart, central and peripheral nervous system, skin, liver, blood, blood vessel, kidney, lung, and pancreas.
